(19) United States
(12) Patent Application Publication
(10) Pub. No.: US 2025/0195051 A1
(43) Pub. Date: Jun. 19, 2025

(54) CONTROL INTERFACES FOR MEDICAL DEVICES

(71) Applicant: REVOLVE SURGICAL INC., Toronto (CA)

(72) Inventors: Matthew Kang, Toronto (CA); Peter Alexander Gordon, Toronto (CA); Peter Francis, London (CA)

(21) Appl. No.: 18/847,037

(22) PCT Filed: Mar. 14, 2023
(86) PCT No.: PCT/IB2023/052466

§ 371 Date: Sep. 13, 2024

Related U.S. Application Data
(60) Provisional application No. 63/319,962, filed on Mar. 15, 2022.

(51) Int. Cl. A61B 17/00; A61B 34/00; A61B 34/30; A61B 50/20

(52) U.S. Cl. CPC A61B 17/00234; A61B 34/30; A61B 34/74; A61B 50/20; A61B 2017/00075; A61B 2017/00207; A61B 2017/00398; A61B 2017/00438; A61B 2017/00442; A61B 2017/00477; A61B 2034/742

(57) ABSTRACT

A control system for a medical device includes: a first interface configured to control a position of the medical device; a second interface installed in a first portion of the handle grip and configured to control an end-effector orientation of an end-effector of the medical device; and a third interface installed in a second portion of the handle grip and configured to control a function of the end-effector. The first interface includes a handle grip coupled to a base member. The base member is coupled to a device attachment unit configured to couple to the medical device. The second interface is communicatively coupled to a sensor assembly that monitors movement detected at the second interface. The third interface is communicatively coupled to the sensor assembly to monitor movement detected at the third interface.

### CROSS-REFERENCE TO RELATED APPLICATIONS

This application claims the priority benefit of U.S. Provisional Patent Application Ser. No. 63/319,962, filed Mar. 15, 2022, the contents of which are herein incorporated by reference in their entirety.

### TECHNICAL FIELD

This disclosure relates generally to the field of minimally invasive surgery (MIS), and more specifically to the field of control systems and interfaces for MIS. Described herein are control interfaces for medical devices.

### BACKGROUND

Surgical procedures can be performed with either an open method, where a large incision is made to access the surgical site, or a minimally invasive surgery (MIS) method, where multiple smaller incisions are made, and slender instruments are used to manipulate tissue at the surgical site. MIS, also known as keyhole or laparoscopic surgery, offers numerous advantages to the patient, such as decreased blood loss, reduced scarring, and reduced length of hospital stay. However, in many cases, the MIS approach is exceedingly difficult to perform, and the open method is implemented instead. A number of causes contribute to the challenges of MIS, but the main difficulties stem from the limitations of the control systems (e.g., interface) for surgical instruments. While many control systems have been developed to address some of the difficulties, conventional control systems still suffer from drawbacks. For example, drawbacks include: having control interfaces that allow instrument articulation with minimal consideration of control interface ergonomics and enabling too many degrees of freedom with a single control interface, resulting in the control of instrument articulation being counterintuitive or even confusing.

Accordingly, there exists a need to develop new control mechanisms that allow improved control for both orientation and position of surgical instruments while maintaining a comfortable and ergonomic control interface to a surgeon.

### SUMMARY

In some aspects, the techniques described herein relate to a control system for a medical device, the control system including: a first interface configured to control a position of the medical device, the first interface including a handle grip coupled to a base member, the base member being coupled to a device attachment unit configured to couple to the medical device; a second interface installed in a first portion of the handle grip and configured to control an end-effector orientation of an end-effector of the medical device, the second interface being communicatively coupled to a sensor assembly to monitor movement detected at the second interface; and a third interface installed in a second portion of the handle grip and configured to control a function of the end-effector, the third interface being communicatively coupled to the sensor assembly to monitor movement detected at the third interface.

In some aspects, the techniques described herein relate to a method of performing a surgical procedure, the method including: actuating, at a first interface of a control system, an input control to position a medical device relative to a remote center of motion; actuating, at a second interface, a first portion of a handle grip configured to control an orientation of an end-effector of the medical device, the second interface being communicatively coupled to a sensor assembly to monitor movement detected at the second interface; and actuating, at a third interface, a second portion of the handle grip configured to control a function of the end-effector, the third interface being communicatively coupled to the sensor assembly to monitor movement detected at the third interface.

In some aspects, the techniques described herein relate to a control system for a medical device, the control system including: a first interface configured to control a position of the medical device, the first interface including a handle grip coupled to a base member, the base member being coupled to a device attachment unit configured to couple to the medical device; a second interface installed in a first portion of the handle grip and configured to control an orientation of an end-effector of the medical device; and a third interface installed in a second portion of the handle grip and configured to control a function of the end-effector.

In some aspects, the techniques described herein relate to a control system for a medical device, the control system including: a first interface configured to control a position of the medical device, the first interface including a handle grip coupled to a base member, the base member being coupled to a device attachment unit configured to couple to the medical device; and a second interface installed in the handle grip and configured to control an orientation of an end-effector of the medical device and a function of the end-effector.

In some aspects, the techniques described herein relate to a control system for a medical device, the control system including: a first interface configured to control a position of the medical device, the first interface including a handle grip coupled to a base member; a second interface installed in a first portion of the handle grip and configured to control an orientation of an end-effector of the medical device, the second interface being communicatively coupled to a sensor assembly to monitor movement detected at the second interface; and a third interface installed in a second portion of the handle grip and configured to control a function of the end-effector, the third interface being communicatively coupled to the sensor assembly to monitor movement detected at the third interface.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings, wherein like reference numerals denote like parts, and in which:

FIG. 1 is a perspective view of an example surgical system attached to a surgical table.

FIG. 2 is a perspective view of an example medical device for use with the surgical systems described herein.

FIG. 3 is a side view of an example robotic surgical system of the surgical system of FIG. 1.

FIG. 4 is a rear isometric view of the example robotic surgical system of FIG. 3.

FIGS. 5A-5B are side views of example implementations of a control system for controlling an example medical device.

FIG. 6 is a rear isometric view of an example control system for controlling an example medical device.

FIG. 7 is a zoomed-in view of an example control configurable with the control systems described herein.

FIG. 8 is an internal perspective view of components configured to actuate the control system described herein.

FIG. 9 is a perspective view of another example control configurable with the control systems described herein.

FIG. 10 is a side view of a user engaging with an example control system with an installed medical device.

FIG. 11 is a perspective view of a user engaging with at least one control of the example control system of FIG. 10.

FIG. 12 is a perspective view of a user engaging with at least one control of the example control system of FIG. 10.

FIG. 13 is a perspective view of a user engaging with at least one control of the example control system of FIG. 10.

FIG. 14 is a perspective view of a user engaging with at least one control of the example control system of FIG. 10.

FIG. 15 is a perspective view of an example control system for controlling an example medical device.

FIG. 16 is a perspective view of an example control system for controlling an example medical device.

FIG. 17 is a perspective view of an example control system for controlling an example medical device.

FIG. 18 is a perspective view of an example control system for controlling an example medical device.

FIG. 19 is a perspective view of an example control system for controlling an example medical device.

FIG. 20 illustrates the internal mechanisms of the example control system of FIG. 19 in trigger-locking mode.

FIG. 21 illustrates the internal mechanisms of the example control system of FIG. 19 in trigger-unlocked mode.

FIG. 22 is a perspective view of an example control system for controlling an example medical device.

FIG. 23 is an example flow diagram of a method of performing a surgical procedure with the control systems described herein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

Some of the challenges of MIS stem from the limitations of the control systems (e.g., interface) for surgical instruments. For example, the control systems for surgical instruments often lack the ability to provide movement dexterity for the surgeon, making it difficult to perform fine tasks, such as suturing, in highly confined spaces. While many control systems have been developed to fit at least some of these criteria, they still suffer from drawbacks, such as balancing instrument articulation with control interface ergonomics. Further, control systems often enable multiple or most degrees of freedom at a single control interface, which makes instrument articulation, via the control interface, counterintuitive or confusing. Accordingly, there exists a need to develop new control mechanisms that at least (1) allow improved control for both orientation and position of medical devices (e.g., surgical instruments), (2) decouple instrument orientation from instrument position for finer, more intuitive instrument control, and (3) maintain a comfortable and ergonomic interface for the user (e.g., a surgeon) using such control systems.

Minimally invasive surgery, in many cases, include processes that can be or should be performed independently, for example, gross positioning of a medical instrument (e.g., a first interface control), robotic assisted actuation of a medical instrument (e.g., a second interface control), and/or actuation of an end-effector of a medical instrument (e.g., a third interface control). The devices and methods described herein are ergonomically and intuitively formulated to reduce or minimize unintended movements of the medical instrument or unintended actuations of the medical instrument. Unintended movements and actuations of medical instruments may result in unintended consequences, which may cause serious ramifications in surgical procedures. As such, ergonomic and intuitive controls may help lower the chances of unintended movements or actuations by increasing usability of controls. Further, ergonomic, intuitive, spatially separated, and/or selective isolation of interface controls may reduce human errors that are a result of a mismatch between expectations of the user and the functions of the interface controls. Still further, increased usability of controls may include the ease of manipulating one interface (e.g., a first, a second, or third) independently of one or more other interfaces. Embodiments described herein may separate, decouple, or lock control interfaces (e.g., first, second, third, etc.) functionally (e.g., manual versus robotically assisted), and/or spatially. For example, the second interface may be spaced apart from the third interface such that interaction with the second interface does not impact or effect movement in the third interface. Similarly, the third user interface may be spaced apart from the second interface such that interaction with the third interface does not impact or effect movement in the second interface. Further, in some embodiments, at least a portion of a stabilizing apparatus may lock (i.e., be selectively isolated or locked) when a second and/or third interface is in use, such that at least a portion of the first interface is not manipulatable or movable when the second and/or third interface is in use. As such, a user of a control system may be less likely to interact with an unintended control interface. Ergonomic and intuitive controls for surgical robotic systems have been contemplated and described herein.

Decoupling position from orientation control may increase or improve familiarity and/or adoption of any of the control systems described herein. For example, users coming from conventional minimally invasive surgery systems may be expecting movements similar to manual instruments that do not engage extra degrees of freedom. As the user becomes comfortable or familiar with the control system, additional degrees of freedom can be engaged and used by the user, for example by interacting with additional interfaces of the control system described herein. For example, when a user is interacting with a first interface, the control interface feels like a manual handle; when the user interacts with the second and/or third interface, the handle actuates robotic-assisted movements which may be intuitive or similar to the interactions with the second and/or third interface controls.

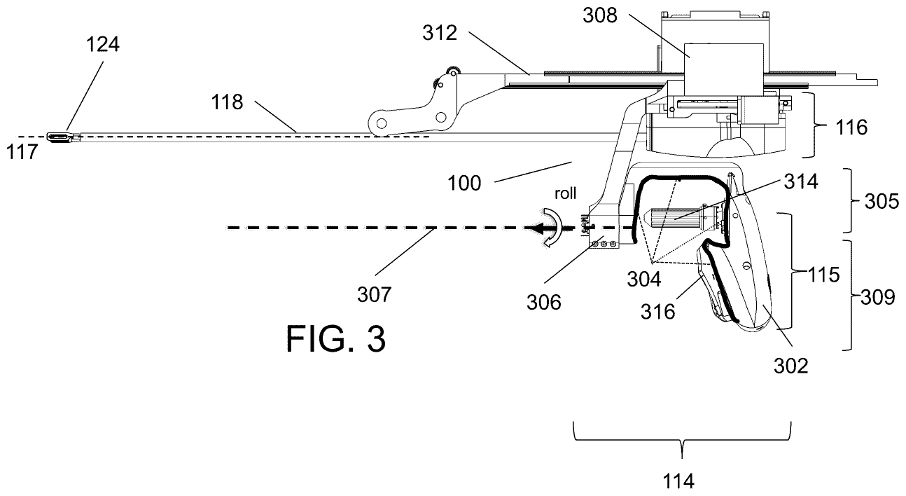

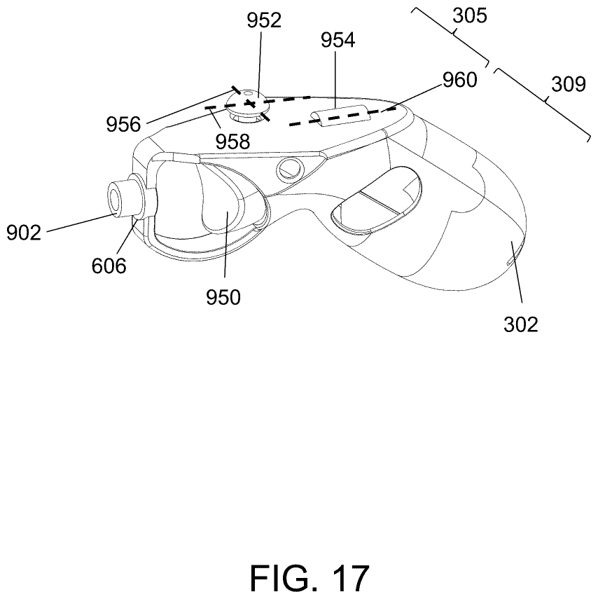

Spatial separation may include positioning interface controls on separate portions of a control system. For example, some of the embodiments described herein position a third interface on a lower portion of an inner grip portion of a handle and a second interface in an upper portion of an inner grip portion of the handle (see, e.g., FIG. 3). These interfaces are spatially separated such that a user likely uses a first one or more fingers to manipulate the second interface and a second one or more fingers to manipulate the third interface. In some embodiments, the second interface is split into two or more interfaces such that roll is separated from pitch and yaw (e.g., FIG. 17); pitch is separated from yaw and roll; or yaw is separated from pitch and roll. Further, engagement of the first interface of some of the embodiments herein may be performed by gripping the handle of the embodiment to apply forces for the gross positioning (and optionally gross roll of the medical instrument) of the embodiment. Additionally, some embodiments separate the roll axis control from the pitch and yaw axis control associated with the second interface (e.g., illustrated in FIG. 17) to further facilitate spatial separation for intuitive control.

Selective isolation and/or locking may include inactivating one or more interfaces while another interface is in use. Alternatively, selective isolation and/or locking can include locking one or more interfaces in an actuated position or unactuated position while another interface is in use.

One factor that can contribute to some of these challenges is the design of the control interface for a medical device (e.g., a surgical instrument) that includes an end-effector, such as a gripper, that is intended to have multiple degrees of freedom. Because conventional minimally invasive surgical instrument control systems couple orientation and positional controls together, it is often difficult to provide controls that are intuitive to a user operating such controls because several degrees of freedom are coupled together. That is, the orientation may inadvertently change when a position is changed because the control system may provide controls that couple the orientation with the position. The systems and methods described herein define a control system with interfaces that allow for reduced cognitive load on a user and an ability to control both orientation and position in a decoupled, but simultaneous fashion.

A unique technical problem arises in conventional minimally invasive surgical instruments (e.g., medical devices) with greater than five degrees of freedom. The technical problem is how to control all available degrees of freedom simultaneously and intuitively. This technical problem is compounded when the medical device (e.g., instrument) is physically connected to a control system. That is, the fulcrum effect occurs during MIS when the instrument is inserted into the surgical site, thereby creating a fulcrum point at the incision. The fulcrum effect results in a distal end of an instrument moving in an opposite direction than the control system or interface (operated by the surgeon) due to the fulcrum point created at the incision. Without appropriate or new control interfaces, the fulcrum effect can render conventional instruments non-intuitive and difficult to use with respect to the motor skills utilized for properly controlling the instruments. The control systems described herein may provide control interfaces for a minimally invasive medical device that provide a technical solution to these technical problems. Further, although the control systems described herein may be described with respect to operation of a system in a fulcrum-effect mode, one of skill in the art will appreciate that the systems described herein may also be used in a system operating in a fulcrum-effect corrected mode (i.e., movement of a proximal control interface is mirrored at a distal end of the instrument).

For example, the control systems described herein may provide intuitive interfaces (e.g., user interface controls) with ergonomic benefits, such as reducing demand on wrists, arms, and/or finger movements performed by the user. In particular, the control systems and associated interfaces described herein may be provided in a handle and/or an associated handle grip (e.g., a handle assembly) to allow a surgeon to control a medical device similar to how the surgeon would control a manual instrument. Some advantages of the described control systems may include the ability to manipulate a dexterous wrist, having relatively improved ergonomics and reduced fatigue as compared to purely manual manipulation of an instrument (e.g., without stabilizing support structures), while also providing a similar level of fine motion control as compared to fully-robotic systems.

While conventional control apparatuses have coupled orientation and position control together, the control systems described herein decouple the position control from the orientation control. The decoupling occurs not only at the apparatus mechanism level but also through user biomechanical actuation of the control system and associated medical device. In particular, the control systems described herein provide for position to be sufficiently controlled by forearm and wrist motions, while orientation is sufficiently controlled by finger motions. The biomechanical actuation of the control systems described herein provides (e.g., for a surgeon) a natural control language that combines inherent surgical training and familiarity with manual surgical tools within minimally invasive surgery.

Further, the control systems described herein provide for a natural extension of existing manual surgical tool interactions translated into a laparoscopic procedure because the control systems allow the use of one or more fingers to control movement of medical devices about the pitch, yaw, and roll axes. The control systems described herein provide an improved solution to the field of minimally invasive surgery (e.g., laparoscopic surgery) by enabling a surgeon to use such a natural control language associated with manual surgical techniques with robotics surgical techniques. In particular, a surgeon may use the control systems and associated user interfaces (mechanical and electromechanical) by using one or more fingers to trigger movements and/or functions of a medical device and/or end-effector.

As used herein, an “end-effector” may include a grasper, a forceps, a scissors, a suturing device, a cutting tool, an ablation element, a cryo-element, a cutting element, a suturing element, a drilling element, milling element, a camera, a needle driver, electro-cautery tool, and the like, such that actuation of the end-effector results in ablation, cryo-activity, cutting, suturing, drilling, milling, electro-cautery, etc.

The term “communicatively coupled” may be defined as either wireless communication (i.e., wirelessly coupled) between components or a wired connection between components.

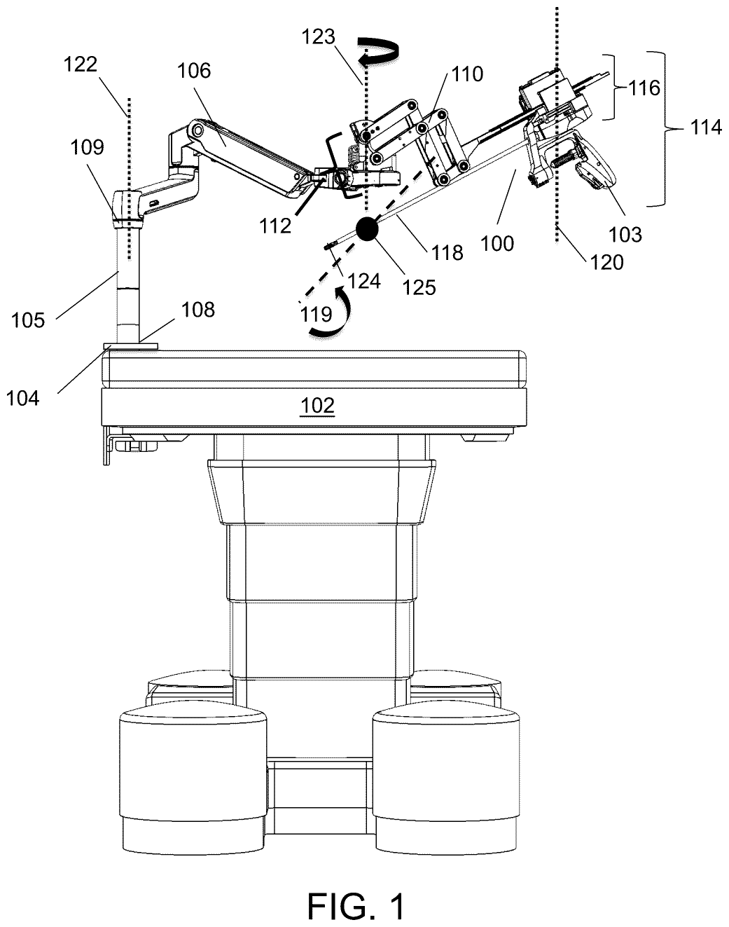

In some implementations, the control system is a handle assembly configured for installation in a hybrid, direct-control and robotic-assisted surgical system, as illustrated in FIG. 1. In operation of such a control system, the handle may provide for control (via one or more interfaces) of a position of an end-effector through articulation of a forearm and/or wrist of the user. The handle may also provide for control (via one or more interfaces) of an orientation of the end-effector through articulation of one or more fingers of the user. While the interfaces used for controlling the position and orientation of aspects of the medical instrument may be operated in a decoupled control mode, the user may operate combinations of the interfaces (e.g., controls) described herein to simultaneously or substantially simultaneously change the position of the medical device (e.g., installed in a portion of the handle) while changing the orientation of an end-effector associated with the medical device. For example, the handle (or handle assembly) described herein may provide a first interface to control a gross positioning of an associated medical device about at least pitch and/or yaw axes and/or insertion/retraction of the associated medical device, for example using a stabilizing apparatus and/or device attachment unit (e.g., a prismatic joint or the like of a device attachment unit). The first interface, in some variations, may also enable rolling of the medical device, about a roll axis. The handle (or handle assembly) described herein may also provide a second interface to control an orientation of an end-effector of the associated medical device about an end-effector roll axis, an end-effector pitch axis, and/or an end-effector yaw axis. The handle (or handle assembly) described herein may also provide a third interface to control one or more functions (e.g., actions) of the end-effector of the associated medical device.

In some embodiments of the control systems described herein, the functionality controlled by the third interface may be combined with the second interface such that there is no third interface. In such embodiments, the second interface controls a distal end of the medical device (e.g., wrist joint and/or end-effector) about the roll, pitch, and/or yaw axes while also controlling a functionality of the distal tip (e.g., grasping, energy delivery, etc.).

In some implementations, the interfaces described herein may be beneficial for hybrid surgical robotic control where the control system is physically connected to the instrument (e.g., medical device) because such interfaces may provide an intuitive movement similar to a conventional laparoscopic handle with the added benefit of mechanical and electromechanical controls that decouple the position from the orientation of the medical device. In general, the systems described herein provide a new control system for a medical device (e.g., a surgical instrument) that allows for scaling and processing of user input into proportional control of an orientation of a robotic end-effector attached to the medical device. Processing of the user input into proportional control allows for motion scaling, limiting, and filtering.

In alternative configurations, the interfaces of various control systems described herein may be purely mechanical (e.g., via cable systems). In other embodiments, any combination of mechanical and electromechanical implementations for any of the interfaces may be employed.

The control systems described herein include a number of user interfaces to provide a plurality of degrees of freedom (DOFs) of movement, for example at least three, four, five, or more degrees of freedom, of the medical device (or a portion of the medical device).

For example, a control system may enable gross mechanical positioning of a medical instrument relative to a base of a stabilizing apparatus, using the various joints of the stabilizing apparatus. Gross mechanical movements of the medical instrument, received at a first interface of a handle (e.g., a grip portion of the handle) and using the stabilizing apparatus, result in a first DOF including movement about a yaw axis of the stabilizing apparatus; and a second DOF including movement about a pitch axis of the stabilizing apparatus. For a third DOF, the medical instrument may be inserted and retracted via a device attachment unit (e.g., via a prismatic joint) and relative to the stabilizing apparatus. For a fourth DOF, the control systems described herein may be rolled about a roll axis to effect a roll movement of the medical instrument. A rolling movement of the control system about a roll axis is with respect to a first attachment point, as discussed in further detail below.

Further, the control systems described herein can include a second interface. The second interface may be in the form of a barrel, joystick control, ring, or the like of a handle having an installed medical device. The second interface may be capable of electro-mechanical movement to provide movement of the end-effector about at least three degrees of freedom. For example, manipulation of the second interface may cause an end-effector of a medical device to: roll about an end-effector roll axis for a first DOF, move about an end-effector yaw axis for a second DOF, and move about an end-effector pitch axis for a third DOF, relative to a resting or unactuated state of the second interface. In some implementations, rolling the second interface results in a proportional rolling of the end-effector about the end-effector roll axis. Although movement about an end-effector roll axis, an end-effector yaw axis, and an end-effector pitch axis are used, one of skill in the art will appreciate that the second interface may be manipulated in any number of intermediate positions between each of the end-effector axes of movement to effect fine wrist joint or medical device tip movements. By manipulating the second interface, a user may be able to control an orientation of an end-effector of the medical device in the at least three degrees of freedom. In addition to robotic surgical systems, surgical devices and systems with manual manipulation of a single instrument with multiple functions or multiple instrument types may benefit from control systems described herein.

In some implementations, the control systems described herein allow for a fourth DOF by enabling control of an end-effector function. This fourth DOF may be enabled by additional functionality at the second interface or by a third user interface.

In some implementations, the control systems described herein allow for movement that provides a wrist movement by actuating the first interface about a roll axis, which has a center of axis offset from the shaft of the medical device. For example, the user may grasp a handle and sweep the handle counterclockwise or clockwise about a pivot point attaching the handle to a control system base. Such a movement rolls the medical device clockwise or counterclockwise about the instrument roll axis.

The control systems described herein include a second interface in the form of a manipulatable control that allows adjustment of an end-effector about an end-effector pitch axis and an end-effector yaw axis. For example, a user can engage the interface and manipulated it about the end-effector pitch axis and the end-effector yaw axis relative to a resting (unactuated) position of the second interface. Such user movements may trigger end-effector movements in corresponding directions and rotationally at angles between such directions. The second interface may allow a roll movement of the end-effector (about a wrist joint of the end-effector) when a user engages the second interface in a twist or spin motion. For example, a twist or spin of the second interface may trigger the end-effector (or a wrist joint of the end-effector or a tip of the medical device) installed in the handle (or associated control system) to roll (e.g., spin on the roll axis of the medical device) in a clockwise or counterclockwise direction according to a particular movement direction selected and performed by the user.

The control systems described herein can include a third interface. The third interface may be in the form of a compressible control. The compressible control may provide an electromechanical actuation of one or more functions of an end-effector of the medical device. For example, the end-effector may have a plurality of predefined functions and such functions may be triggered by a user interacting with the compressible control. Example functions include, but are not limited to, closing of mechanical jaws, administration of electrical energy, administration of cryotherapy, cutting motions, grasping motions, a material removal function (e.g., cutting, drilling, deburring, etc.), an irrigation function, a suction function, a stapling or adhesive function, etc.

In some implementations, the third interface may take the form of one or more buttons, a touchpad, a ring, a lever, a compressive portion of a barrel or joystick (described above), or an actuator including one or more sensors that allow for continuous actuation and latching of the third interface at a maximum actuation point. This latching may provide for maximum actuation of the medical device (e.g., the end-effector function(s)) without continuous interaction from the user. For example, the function may provide for sustained grasping of human tissue without continued interaction with the third interface.

FIG. 1 is a perspective view of an example robotic surgical system 100 attached to a patient support apparatus 102. The robotic surgical system 100 includes robotic-assisted components as well as manually movable components. In short, the system 100 includes at least a handle 103 coupled to a control system 114 that is configured to manipulate a medical instrument 118 and/or end-effector 124 associated with the medical instrument 118. The end-effector 124 is manipulated by a user (e.g., a surgeon) via one or more interfaces (e.g., handle grip, handle assembly, etc.) of the control system 114. Various interfaces of the handle 103 may be manipulated by one or more fingers of a user (e.g., a surgeon).

As illustrated, the robotic surgical system 100 includes a remote base 104 that provides stabilization for a support arm 105 and pivotable arm 106. The remote base 104 is attached to a patient support apparatus 102 at a first attachment point 108 and attached to a first end of the support arm 105. Although the attachment point is illustrated attached to a surface of the patient support apparatus 102, other implementations may clamp to attachment points that are rails, slats, raised platforms, a ceiling system or rail, and the like. The attachment of the remote base 104 to the patient support apparatus 102 may be achieved in a number of ways, such as a clamping mechanism, a bolting system, or the like. The patient support apparatus may be either manufactured with connection points or a connection method specifically for the attachment of the base 104, or the base 104 may be designed such that it can be attached to any existing operating table or patient support apparatus.

A second end of the support arm 105 is attached to a first end of the pivotable arm 106 at a second attachment point 109. A second end of the pivotable arm 106 is attached to a first end of a stabilizing apparatus 110 (e.g., including a parallelogram movement mechanism) at an attachment point 112. In some implementations, attachment point 112 includes a revolute joint, which enables movement of the medical instrument about a yaw axis 123 of stabilizing apparatus 110. The stabilizing apparatus 110 enables a second DOF of the medical instrument about a pitch axis 119 of the stabilizing apparatus.

The robotic surgical system 100 also includes a control system 114. The control system 114 is attached to the stabilizing apparatus at a second end of the stabilizing apparatus 110. The control system 114 includes user interfaces (described in detail below), electronics (described in detail below), and a device attachment unit 116 that is removably attached to a medical instrument 118. The support arm 105, base 104, and/or stabilizing apparatus 110 may function to support (or partially support) the weight of the control system 114 and/or any installed medical device(s). For example, the stabilizing apparatus 110 may define a remote-center-of-motion (i.e., at pivot point or insertion point 125). The remote base 104 is configured to be fixed relative to a patient. The stabilizing apparatus 110 is attached to the device attachment unit 116. The device attachment unit 116 is movable relative to the stabilizing apparatus 110, and ultimately the remote base (e.g., base 104), and is configured to removably receive the control system 114. In some implementations, the remote base 104 may define a first positional reference frame 122 while a portion of the control system 114 may define a second positional reference frame 120.

In operation, the control system 114 and one or more interfaces of the handle 103 allow for pitch, yaw, and roll control of the distal wrist joint of the end-effector 124. Such a configuration provides for orientation control of the end-effector 124 of the medical instrument 118. The control system 114 provides a number of interfaces used to orient the end-effector 124 about end-effector pitch, roll, and/or yaw axes. The movements received at the one or more interfaces may result in proportional movements of the end-effector 124.

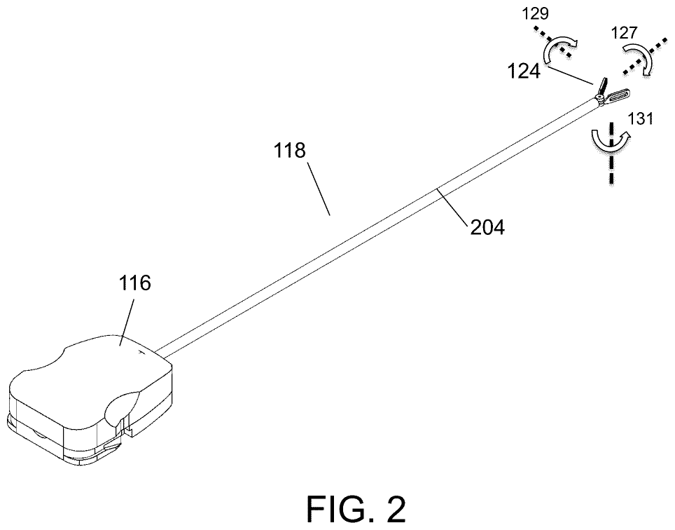

FIG. 2 is a perspective view of an example medical instrument 118 for use with the surgical systems described herein. The medical instrument 118 may be a surgical instrument that is removably attachable to a device attachment unit 116. For example, the medical instrument 118 may be a surgical device with an elongate body 204 extending from a distal tip that includes the end-effector 124.

The attached medical devices described herein (e.g., medical instrument 118) have a wristed end-effector 124 at their tips with at least three degrees of freedom, such as pitch, yaw, and roll capabilities. The attached medical devices described herein may also include grasping and/or other or additional end-effector actuation to provide a surgeon with an increased degree of manipulation of the end-effector 124 and/or medical instrument 118 at the surgical site as compared to a non-wristed medical device. As it can be difficult to control multiple degrees-of-freedom of an end-effector mechanically, the device attachment unit 116 includes a controller and a powered actuation unit that is configured to control the orientation of the end-effector 124 of the medical instrument 118.

For example, the device attachment unit 116 may include electronics (not shown) therein. In some implementations, the device attachment unit 116 may provide motorized units that may be operatively coupled to the medical instrument 118. The device attachment unit 116 is configured to receive an elongate body 204 of the medical instrument 118. The device attachment unit 116 is also configured to receive mechanical and electronic commands via control system 114. In some implementations, the powered actuation unit may reside in the device attachment unit 116. In some implementations, the powered actuation unit may reside in a housing or control housing or box for the device attachment unit 116.

The control system 114 may include at least a controller communicatively coupled to a sensor assembly to receive corresponding sensor signals and to generate a corresponding primary control signal. In addition, the control system 114 may include the powered actuation unit communicatively coupled to the controller to receive each primary control signal. The powered actuation unit is configured to actuate the end-effector 124 of the medical instrument 118, based on each received primary control signal when the medical instrument 118 is received (e.g., installed) in the device attachment unit 116.

As illustrated in FIG. 2, the medical instrument 118 may be operated via control system 114 to orient an end-effector about the end-effector roll axis 127, end-effector pitch axis 129, and/or end-effector yaw axis 131. The end-effector 124 may be a grasper, a sensorized end-effector, a force-torque sensor, a material removal tool (e.g., cutting, drilling, deburring, etc.), a welding torch, a collision sensor, a tool changer, a laser, a hook, a cautery/electrosurgery tip, a clip applier, a needle driver, a scissors, an ultrasonic energy instrument, an irrigation tip, a vessel sealer, a stapler, a cryo-ablation tool, just to name a few examples. The end-effector 124 may be configured to perform example functions including, but not limited to, opening/closing of mechanical jaws, administration of electrical energy, cutting motions, grasping motions, a material removal function (e.g., cutting, drilling, deburring, etc.), an irrigation trigger, a stapling or adhesive trigger, just to name a few examples.

FIG. 3 is a side view of an example robotic surgical system 100 of the surgical system of FIG. 1. The system 100 includes a handle 302 with an inner portion handle grip 304 and a palm grip on an outer portion 115. In some implementations, the handle grip 304 is pivotally attached to the base member 306. For example, rotation of handle 302 or handle grip 304 relative to base member 306 effects rotation of a shaft or elongate body of the medical instrument about an instrument roll axis 117. The handle 302 (with the handle grip 304) is coupled to a base member 306, which is electrically coupled to circuitry and motors 308 for operating portions of the system 100. The base member 306 is also electrically coupled to at least a portion of device attachment unit 116 (also described herein as a control interface) to interface with mounting arm 312, which may include a rail, prismatic joint, rod, bar, rack, or the like for translation of the device attachment unit 116 relative to mounting arm 312. The mounting arm 312 is configured to couple to stabilizing apparatus 110. The base member 306 is also electrically coupled to the device attachment unit 116. The device attachment unit 116 is configured to receive the medical instrument 118 therein to effect movements (positional, orientational, etc.) of the medical instrument 118.

In some implementations, the handle grip 304 is movable relative to the device attachment unit 116 about at least a first degree of freedom, for example to roll the medical instrument 118 about a roll axis 117. The handle grip 304 may also function as a first interface, which may be used by the user to determine and set an orientation of the handle grip 304 about the first degree of freedom.

As illustrated, the handle grip 304 is configured with a first interface that provides for mechanically (but could also be electromechanically implemented) based movement of the control system 100, using the various joints of the stabilizing apparatus 110 (illustrated in FIG. 1). The first interface is configured to control a position of the medical device. In some implementations, the first interface can be used to position the medical instrument 118 (and/or a distal tip of end-effector 124) into a particular position using a user-selected positional reference frame. For example, the user may move the system 100 in space to position the medical instrument 118 by maneuvering the first interface (e.g., the handle 302) using one or both of a forearm and a wrist (not shown) to cause movement of the medical device. In this example, the first interface comprises the handle 302 configured to receive a grip of a user. The user may then provide movement of system 100 using the handle 302 (and/or handle grip 304).

For example, a user may move the system 100 about one or both of the pitch axis 119 (illustrated in FIG. 1) and yaw axis 123 (illustrated in FIG. 1), illustrated as movement about an insertion point 125 of the medical instrument 118 into a surgical site. Further, for example, the first interface may be configured to manipulate the medical instrument 118 about a roll axis 117. The first interface may receive the movement as a first input to position the medical instrument 118 relative to a predefined positional reference frame, for example a remote center of motion comprising pivot point or insertion point 125. In some implementations, the predefined positional reference frame may be associated with the base member 306, as illustrated by positional reference frame 120 in FIG. 1. In some implementations, the predefined positional reference frame may be associated with the base 104 of arm 105, as illustrated by positional reference frame 122 in FIG. 1.

Therefore, as illustrated in FIGS. 1 and 3, when actuating the first interface, the user maneuvers the first interface using one or both of a forearm and a wrist to cause movement of the medical instrument 118 about a pitch axis 119, yaw axis 123, and/or a roll axis 117 associated with the selected positional reference frame. In this example, the forearm or wrist align with an offset axis 307 to the medical instrument 118 shaft. The positional reference frame is selectable by the user. In addition, the weight of the system 100 need not be supported by the user's hand grip, due to the counterbalancing performed by the stabilizing apparatus 110. Counterbalancing the weight of the system 100 provides an ergonomic advantage for the user because the user may be free to grip the handle 302 and begin moving naturally in space with the arm, forearm, wrist, fingers, etc. without having to hold the entire weight of the system 100. The location (and selected positional reference frame) of a wrist and/or forearm axis of movement and/or rotation may be selected or otherwise configured by the user. In general, the offset axis 307 of system 100 is configured to be aligned with an anatomically natural arm movement of the user.

Illustrated in FIG. 3, the handle grip 304 is configured with a second interface, illustrated here as a joystick 314. In this example, the second interface (e.g., joystick 314) is installed in a first portion 305 of the handle grip 304. The first portion 305 is an upper portion of the handle grip 304. The second interface (e.g., joystick 314) is configured to control an orientation of the end-effector 124 of the medical instrument 118.

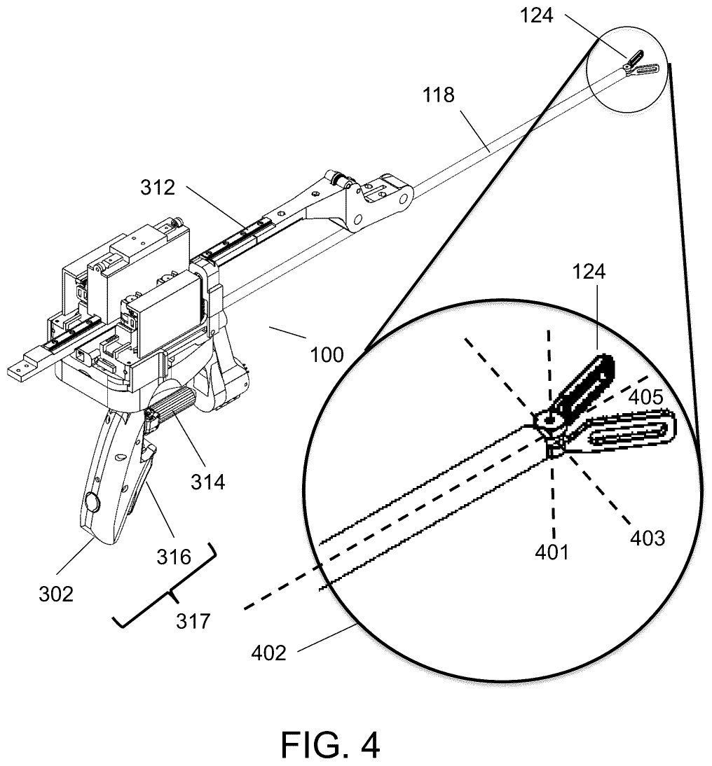

The second interface may provide control inputs for electromechanical movement for system 100. For example, the second interface may be communicatively coupled to a number of electronics, sensors, and/or motors that allow a user to use the second interface (e.g., via the joystick 314) to change the orientation of the end-effector 124, as illustrated in detail in FIG. 4. In other implementations, the second interface may be purely mechanical, for example by controlling one or more pull wires, concentrically bonded tubes, or the like to control orientation of the end-effector 124 or distal tip.

In some implementations, the second interface includes a tiltable and/or rotatable joystick finger interface. A user may actuate the second interface by manipulating the joystick 314 of the handle grip 304 with one or more fingers, for example a thumb and a finger in some implementations. Movements performed at the second interface (e.g., joystick 314) may cause a change in orientation of the end-effector 124 or distal tip about a first axis 403 (e.g., end-effector pitch axis), a second axis 401 (e.g., end-effector yaw axis), and/or a third axis 405 (end-effector roll axis). For example, the second interface (e.g., joystick 314) includes an electromechanical controller subsystem configured to receive and translate inputs (produced by the second interface) to articulate the end-effector 124 about the first axis 403 (e.g., a pitch axis), the second axis 401 (e.g., a yaw axis), and the third axis 405 (e.g., a roll axis). Although the second interface (e.g., joystick 314) is illustrated as being positioned along an inner surface or inner grip portion of handle 302, one of skill in the art will be appreciate that the second interface (e.g., joystick 314) may be positioned along an outer grip or palm portion of handle 302, along a lower portion 317 of handle 302, for example in place of third interface (e.g., compressible control 316), or otherwise for ergonomic movement of the second interface (e.g., joystick 314).

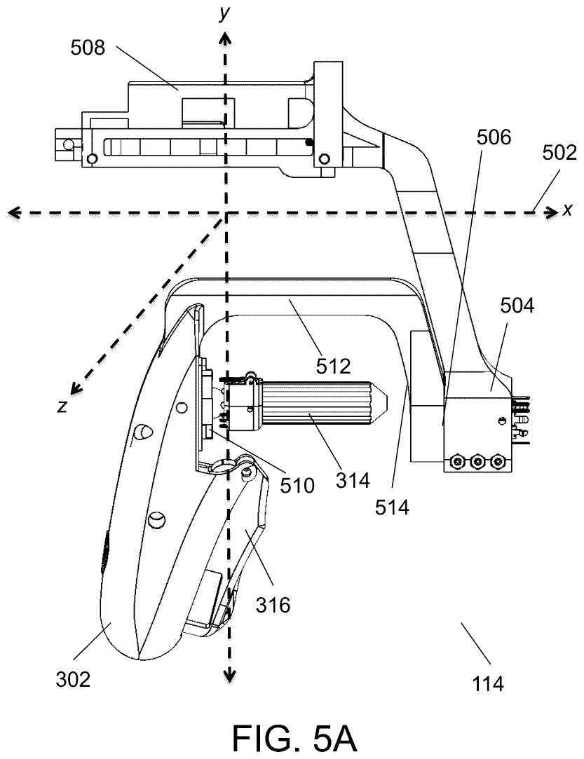

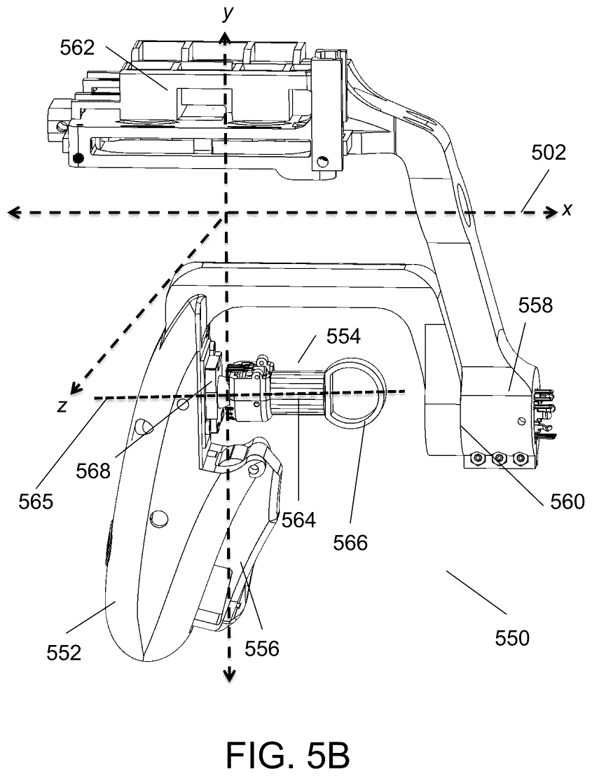

In some implementations, the second interface includes a ringed finger interface portion (e.g., ring portion 566 of FIG. 5B). For example, and as illustrated in FIG. 5B, instead of a joystick, the second interface may be a ring that is movable about three degrees of freedom within the handle grip. The ring may function similar to the joystick in that one or more fingers may control the orientation of the end-effector 124. However, a ring shape may allow the user to grasp the second interface with a thumb and a finger and may allow the user to place a finger within the ring to articulate and/or actuate the end-effector 124. Other interfaces are of course possible including, but not limited to touchpads, buttons, levers, switches, toggle switches, trackball, or the like. These other interfaces may control movement of any one of the medical devices or device portions described herein. In some implementations, other interfaces may be installed on the control systems described herein to control functions of the medical device and/or end-effector including, but not limited to braking, electrocautery, adjustment or locking of wrist positions, and the like.

In some implementations, the second interface is communicatively coupled to a sensor assembly to monitor movement detected at the second interface (e.g., the joystick, ring, etc.). For example, the second interface (e.g., the joystick 314, ring portion, etc.) may be connected to one or more electrical cables and a wireless or wired communication protocol. In some implementations, the sensor assembly may be configured to monitor a position of a handle, second interface (e.g., joystick 314), a trackball, a triggering of a switch or button, a pressure applied to a pressure sensitive sensor, and the like using suitable sensors. The sensor assembly may be configured to use the monitoring to trigger generation of a corresponding sensor signal.

A sensor signal, associated with one or more of the interfaces described herein, can be provided to a suitable controller (that may be a computer, PLC, microprocessor, and the like) that can receive the sensor signal and generate a corresponding control signal that is appropriate for the medical instrument 118 and/or end-effector 124 (illustrated in FIG. 4). The control signal is provided to a powered actuation unit that is communicatively coupled to the controller and configured to cause movement of the medical instrument 118. For example, the powered actuation unit is configured to engage and position the medical device and/or orient the end-effector 124 of the medical instrument 118 based on the user inputs provided at one or more interfaces. The powered actuation unit may then mimic the inputs from the user into corresponding actions/outputs by the end-effector 124 and/or medical instrument 118. In some implementations, the sensor assembly is configured to generate a corresponding sensor signal based on a detected input at the second interface (e.g., the joystick 314, ring portion, etc.) or a detected input at the third interface (e.g., compressible control 316).

As illustrated in FIGS. 3 and 4, the handle grip 304 is configured with a third interface, illustrated as a compressible control. The third interface (e.g., compressible control 316) is illustrated installed in a second portion 309 of the handle grip 304. The second portion 309 of the handle grip 304 may include a middle and lower portion 317 of the handle grip 304. The third interface (e.g., compressible control 316) may be configured to control a function of the end-effector 124. For example, the third interface (e.g., compressible control 316) may be configured to receive and translate a trigger input provided by a user to control the function of the end-effector 124 and/or to maintain a state of the end-effector. That is, if the user actuates the third interface (e.g., compressible control 316) by gripping the third interface (i.e., activating the compressible control 316) with one or more fingers, for example, the end-effector 124 may perform a predefined function and may then remain in the state until the user performs another action. In some implementations, the other action is releasing or compressing the third interface (e.g., compressible control 316) to terminate the predefined function. In some implementations, the other action is increased compression of the third interface (e.g., compressible control 316). In some implementations, the other action may include activation of another control, interface, button, and/or movement. In some implementations, the third interface (e.g., compressible control 316) may be configured to control a plurality of functions of the end-effector 124. The plurality of functions may be performed sequentially, concomitantly, or substantially simultaneously.

In some implementations, the third interface (e.g., compressible control 316) may be communicatively coupled to the sensor assembly (similar to the second interface) to measure movement at the third interface (e.g., compressible control 316). The sensor assemblies described herein may include at least one potentiometer (e.g., rotary or linear) or encoder (e.g., linear or rotary) to detect an orientation of the component or interface housing the sensors of the particular assembly. For example, the sensor assemblies described herein may include one or more potentiometers or encoders to detect the orientation of the second interface (e.g., the handle 302 about at least one of the pitch, roll, and yaw axes; and/or a state (e.g., activated vs. deactivated, compressed vs. decompressed, etc.) of the third interface (e.g., compressible control 316). Alternatively, the third interface may be purely mechanical (not electromechanically controlled).

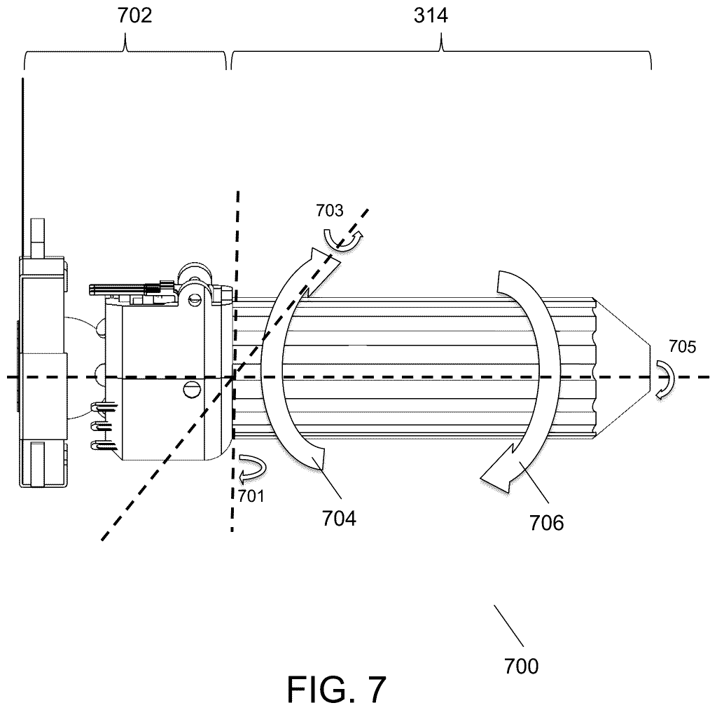

FIG. 4 is a rear isometric view of the example robotic surgical system 100. In this example, the system 100 is mounted on the mounting arm 312 (e.g., rail, prismatic joint, bar, rack, rod, etc.), which may be mounted onto a larger surgical infrastructure, as described in FIG. 1. The first interface (e.g., handle 302, handle assembly, grip portion, etc.) includes the second interface (e.g., joystick 314) and the third interface (e.g., the compressible control 316) mounted on the first interface (e.g., the handle 302). A zoomed-in view 402 of the end-effector 124 is illustrated. The view 402 depicts the end-effector pitch axis 403, the end-effector roll axis 405, and the end-effector yaw axis 401. FIG. 7 illustrates how various axes of movement of the second interface (e.g., joystick 314) map to roll, yaw, and pitch orientation of the end-effector 124 (as illustrated in FIG. 4). For example, the second interface (e.g., joystick 314) may be manipulated about a roll axis 705, a pitch axis 703, and a yaw axis 701 to effect movement of the end-effector about the end-effector pitch axis 403, the end-effector roll axis 405, and the end-effector yaw axis 401, as illustrated in FIGS. 4 and 7. Of course, one of skill in the art will appreciate that nearly infinite intermediate positions between each of the illustrated axes of movement are possible with the control systems described herein. For example, intermediate positions between a first roll limit and a second roll limit, a first pitch limit and a second pitch limit, or a first yaw limit and a second yaw limit are contemplated herein.

In operation, a user may operate the handle 302 to engage with the tiltable and rotatable second interface (e.g., joystick 314) and/or the third interface (e.g., compressible control 316). To actuate the second interface and cause movement of the end-effector 124, the user may move the second interface (e.g., joystick 314) by gripping the second interface with one or more fingers, for example a thumb and a finger. Movements performed at the second interface (e.g., joystick 314) may cause a change in orientation of the end-effector 124 about a first axis 403 (e.g., pitch axis), a second axis 401 (e.g., yaw axis), and/or a third axis 405 (roll axis), as illustrated in zoomed-in view 402. Actuating the third interface (e.g., compressible control 316) and causing movement and triggering one or more functions of the end-effector 124, may include the user gripping the third interface. In some implementations, the third interface (e.g., compressible control 316) includes one or more additional controls such as trackpad, buttons, switches, and the like. For example, the third interface (e.g., compressible control 316) may function to lock a wrist in a selected orientation, to lock an end-effector in a selected configuration, to lock the handle in a selected orientation, to zero an orientation of the wrist, etc.

FIG. 5A is a side view of an example control system 114 for controlling an example medical instrument 118. For example, the control system 114 may include a first interface as an exemplary, non-limiting handle 302, a second interface illustrated as an exemplary, non-limiting joystick, and a third interface illustrated as an exemplary, non-limiting compressible control 316. Additional interfaces may be configured for control system 114 based on which medical device is configured for use with the control system 114. Further, the functions of the second interface (e.g., a joystick 314) and the third interface (e.g., a compressible control 316) may be merged into a single interface or split into additional interfaces.

The handle 302 (and/or handle grip) is configured as a mechanical interface for receiving input from a forearm, wrist, and/or hand of a user (i.e., a first interface). For example, the user may use a forearm or hand to engage with handle 302 to provide input (e.g., grasp, twist, push, pull, lift, lower, and/or rotationally move) to position an installed medical device relative to a predefined positional reference frame, as illustrated by axis 502 or relative to a remote center of motion (shown as pivot point or insertion point 125, illustrated in FIG. 1 and described above). The axis 502 may move with the control system 114 as the user moves the handle. The handle 302 is illustrated attached to a base 504 at a pivot point 506.

The second interface (e.g., joystick, barrel, etc.) is configured as an electromechanical interface for receiving finger input. For example, the user may use one or more fingers to engage with second interface (e.g., the joystick 314 described for FIGS. 3, 4, 5A, 6 and 7) to provide input (e.g., adjust orientation of an end-effector about an end-effector pitch, yaw, and/or roll axis) to select an orientation for an end-effector of the medical device (not shown). The second interface (e.g., joystick 314) may be configured to interface with a control housing 508 to receive a plurality of inputs and have those inputs translated into motion of the end-effector associated with the control system 114. The control housing 508 may include cables, electrical assemblies, motor assemblies, processing assemblies, electromechanical controller subsystems, etc. that are wired to receive and translate input movements at the second interface to be translated to trigger articulation of the medical device and/or end-effector around the selected (e.g., about an x-axis, a y-axis, and/or a z-axis) and/or predefined axis 502.

By way of a non-limiting example, a user or a robotic interface may actuate the first interface (e.g., the joystick 314 as described for FIGS. 3, 4, 5A, 6 and 7) by providing a first input. The first input may be a manual input. For example, the manual input may be an input performed by a user as a mechanically actuated input with the handle 302. In some implementations, the manual input may be an input performed by a robotic interface that provides a mechanically actuated input with the handle 302. The input may be a mechanical input providing movement of the control system (or a component thereof) that is coupled to the medical instrument 118 without engaging electromechanical components of the control system.

In some implementations, the plurality of inputs provided at the second interface include motions received at the second interface (e.g., joystick 314). The motions may be performed by fingers of the user to engage electromechanical components (e.g., sensorized joints) to orient the end-effector 124. In some implementations, the motions may be performed by a robotic interface to engage electromechanical components to orient the end-effector 124.

In some implementations, the trigger input includes one or more finger inputs received at the finger interface of the third interface (e.g., the compressible control 316 described for FIGS. 3, 4, 5A, 6 and 9). The one or more finger inputs may be configured to engage additional electromechanical components to perform a predefined function of the end-effector 124. In some implementations, the one or more finger inputs may be performed by a robotic interface to engage additional electromechanical components to perform a predefined function of the end-effector 124.

In some implementations, the control housing 508 includes components for detecting sensor signals from one or more of the sensor assemblies described herein. The sensor signals may be translated and provided to components of the control housing 508 in order to change the position and/or orientation of the end-effector about at least one of the pitch, roll and yaw axes.

In some variations, the second interface (e.g., a joystick 314 FIGS. 3, 4, 5A, 6 and 7) also includes a switch 510 that provides electromechanical interaction between one or more sensors (not shown) associated with the second interface (e.g., joystick 314) and other components of the control system 114. In particular, the switch 510 may allow the second interface to pivot within the handle grip. In some implementations, the switch 510 is also communicatively coupled and electrically coupled to components in the control housing 508 to enable transfer of signals between the input mechanisms of control system 114 and the outputs affecting orientation of the end-effector and/or movement of the medical device.

Although the second interface (e.g., the joystick 314 as described for FIGS. 3, 4, 5A, 6 and 7) in the handle grip associated with handle 302 is depicted substantially perpendicular to the y-axis, the second interface may be installed within the handle grip at any angle. For example, the second interface (e.g., joystick 314) may be installed in the handle grip from about zero to about fifteen degrees in the x-y axis. In some implementations, the second interface (e.g., joystick 314) may be installed at about −5 degrees to about −15 degrees in the x-y axis. In some implementations of the control system 114, the second interface (e.g., joystick 314) may be installed in other locations within the handle grip. For example, the second interface (e.g., joystick 314) may instead be installed in an arm portion 512 with the switch 510 installed in the arm portion 512 and the second interface (e.g., a joystick 314) installed substantially parallel to the y-axis. In another example, the second interface (e.g., a joystick 314) may be installed in an arm portion 514 with the switch 510 installed in the arm portion 514 and the second interface (e.g., a joystick 314) installed to be substantially parallel to the x-axis. In such a configuration, the user may use a thumb to control the second interface (e.g., a joystick 314) to trigger movement of the end-effector about the pitch and/or yaw axes (see also FIG. 4). The user may also use the second interface (e.g., a joystick 314) in such a configuration by using one or more fingers and/or a thumb to control the end-effector about the roll axis (see also FIG. 4).

The third interface (e.g., compressible control 316) is configured as a compressible finger interface configured to receive and translate a trigger input to control the function of the end-effector of the medical device and/or to maintain a state of the end-effector, as described in detail above. In some implementations, the compressible control input is provided in another location of control system 114 to enable a thumb or other finger to engage the third interface. For example, the third interface (e.g., compressible control 950) of the control system embodiment of FIG. 17 is located in first portion 305 the handle 302, and is suitable for index finger actuation.

In some implementations and as illustrated by FIG. 1, the interfaces (e.g., first, second, and third) described herein may be configured to provide haptic feedback to a user that is engaged with handle 302 and using one or more of the interfaces of control system 114. For example, the control system 114 may be configured to sense that an installed medical instrument (e.g., medical instrument 118) or the end-effector (e.g., end-effector 124) is in a particular condition such as being over torqued, beyond a predefined limit, experiencing restricted movement (i.e., snagged on something), moving above a threshold speed, or other feedback associated with a surgical procedure and the equipment being used. If a forementioned condition is met, the control system 114 may trigger a particular component (e.g., handle 302, joystick 314, and/or compressible control 316) to provide haptic feedback to the hand and/or fingers of the user operating the installed medical device.

FIG. 5B is a side view of another example control system 550 for controlling an example medical instrument 118 (not shown). For example, the control system 550 includes a handle 552 functioning as a first interface, a joystick 554 functioning as a second interface, and a compressible control 556 functioning as a third interface. Additional interfaces may be configured for control system 550 based on which medical device is configured for use with the control system 550.

The handle 552 (and/or handle grip) is configured as a mechanical interface for receiving input from a forearm, wrist, and/or hand of a user. For example, the user may use a forearm or hand to engage with handle 552 to provide input (e.g., grasp, twist, push, pull, lift, lower, and/or rotationally move) to position an installed medical device relative to a predefined positional reference frame, as illustrated by axis 502. The axis 502 may move with the control system 550 as the user moves the handle. The handle 552 is illustrated attached to a base 558 at a pivot point 560. Such positional movements, received at a first interface (e.g., handle 552), are described in greater detail in connection with FIG. 1.

The joystick 554 is configured as an electromechanical interface for receiving finger input. For example, the user may use one or more fingers to engage with joystick 554 to provide input (e.g., adjust orientation of an end-effector about a pitch, yaw, and/or roll axis) to select an orientation for an end-effector of the medical device (see also FIG. 4). The joystick 554 may be configured to interface with a control housing 562 to receive a plurality of inputs and have those inputs translated into motion of the medical device and/or end-effector associated with the control system 550. The control housing 562 may include cables, electrical assemblies, motor assemblies, processing assemblies, electromechanical controller subsystems, etc. that are wired to receive and translate input movements at the joystick 554 to be translated to trigger articulation of the medical device and/or end-effector around the selected (e.g., end-effector pitch, yaw, and/or roll axis) and/or predefined axis 502 (see also FIG. 4).

By way of a non-limiting example, a user or a robotic interface may actuate the first interface (e.g., handle 552) by providing a first input. The first input may be a manual input. For example, the manual input may be an input performed by a user as a mechanically actuated input with the handle 552. In some implementations, the manual input may be an input performed by a robotic interface that provides a mechanically actuated input at the handle 552. The input may be a mechanical input providing movement of the control system (or a component thereof) that is coupled to the medical instrument 118 without engaging electromechanical components of the control system.

In some implementations, the plurality of inputs provided at the second interface include motions received at the joystick 554. The motions may be performed by fingers of the user to engage electromechanical components to orient the end-effector 124, for example. In some implementations, the motions may be performed by a robotic interface to engage electromechanical components to orient the end-effector 124. Such motions will be described in greater detail in connection with FIG. 7.

In some implementations, the third interface (e.g., compressible control 556 or trigger input) includes one or more finger inputs received at the finger interface. The one or more finger inputs may be configured to engage additional electromechanical components to perform a predefined function of the end-effector 124. In some implementations, the one or more finger inputs may be performed by a robotic interface to engage additional electromechanical components to perform a predefined function of the end-effector 124. The one or more finger inputs may engage with a barrel portion 564 of the joystick 554 and/or a ring portion 566. For example, the user may use a thumb and a forefinger to engage with ring portion 566 to turn the joystick 554 to trigger an end-effector 124 to move or otherwise perform a function. In some implementations, the user may use at least one finger to engage the ring portion 566 to move the joystick 554 (e.g., about an end-effector pitch, yaw and/or roll axis). Actuation of the ring portion 566, in some embodiments, may be decoupled from actuation of the barrel portion 564. For example, manipulation of the ring portion 566 may not affect actuation of the barrel portion 564 or manipulation of the barrel portion may not affect actuation of the ring portion 566. As such, a user may manipulate the ring portion 566 independently of the barrel portion 564 and vice versa. In one exemplary, non-limiting embodiment, the ring portion 566 is manipulated using one finger and the barrel portion 564 is manipulated using a second, different finger. The barrel portion 564, depicted as being ergonomically positioned for thumb and/or finger actuation, may include a potentiometer (or encoder), which measures a roll orientation about the roll axis 565. The measured roll orientation may be received by the control system to roll the end-effector in the same direction. For example, if the barrel portion 564 is rolled to the left, the end-effector 124 would be rolled to the left about the roll axis 405 (illustrated in FIG. 4). Further, the end-effector may be rolled while being pitched and/or yawed. As such, a rolled end-effector 124 actively being pitched and/or yawed would roll about a roll axis offset by the same amount of pitch and/or yaw as the manipulated end-effector.

In some implementations, the control housing 562 includes components for detecting sensor signals from one or more of the sensor assemblies described herein. The sensor input may be translated and provided to components of the control housing in order to change the position and/or orientation of the end-effector about at least one of the pitch, roll, and yaw axes.

The joystick 554 also includes a joystick switch 568 that provides electromechanical interaction between one or more sensors (not shown) associated with the joystick 554 and other components of the control system 550. In particular, the switch 568 may allow the joystick 554 to pivot within the handle grip. In some implementations, the switch 568 is also communicatively coupled and electrically coupled to components in the control housing 562 to enable transfer of signals between the input mechanisms of control system 550 and the outputs affecting articulation of the end-effector and/or movement of the medical device.

Although the joystick 554 in the handle grip associated with handle 552 is depicted substantially perpendicular to the y-axis, the joystick 554 may be installed within the handle grip at any angle. For example, the joystick 554 may be installed in the handle grip from about zero to about fifteen degrees in the x-y axis. In some implementations, the joystick 554 may be installed at about −5 degrees to about −15 degrees in the x-y axis. In some implementations of the control system 550, the joystick 554 may be installed in other locations within the handle grip.

The third interface (e.g., compressible control 556) is configured as a compressible finger interface configured to receive and translate a trigger input to control the function of the end-effector of the medical device and/or to maintain a state of the end-effector, as described in detail above. In some implementations, the trigger input is provided in another location of control system 550 to enable a thumb or other finger to engage the third interface (e.g., compressible control 556). For example, the third interface (e.g., compressible control 950) of the control system embodiment of FIG. 17 is located in first portion 305 the handle 302, and is suitable for index finger actuation. In any of the embodiments described herein, a compressible control may be replaced with first and second paddles, for example, that may be squeezed together or separated to effect a function of an end-effector.

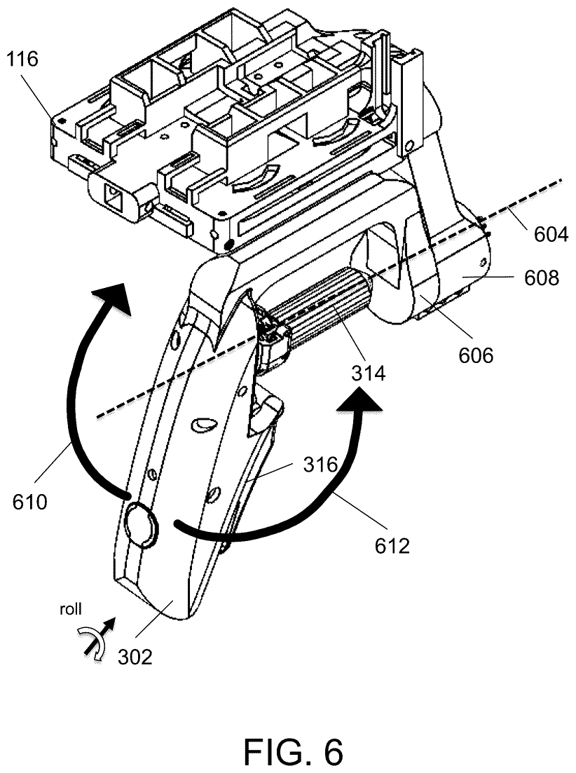

FIG. 6 is a rear isometric view of an example control system for controlling an example medical device. For example, the control system illustrated in FIG. 6 may represent the control system 114 (illustrated in FIG. 5A) with all or a portion of the control housing 508 removed to provide a view of the device attachment unit 116. The device attachment unit 116 includes at least one aperture and/or connection portion in which to receive and hold a medical device. The device attachment unit 116 may be held over a patient during a surgical procedure by stabilizing apparatuses including, but not limited to arm 312 (e.g., prismatic joint) connected to stabilizing apparatus 110 (e.g., including a parallelogram movement mechanism), pivotable arm 106, support arm 105 and base 104 (illustrated in FIGS. 1 and 3). A user (e.g., a surgeon) may directly control the device attachment unit and the attached medical device via control system 114 and handle 302.

In operation, a user may grasp the handle 302 and may move the handle about a pivot centerline or instrument roll axis 604. In particular, a handle portion 606 may pivot about a base portion 608 in the direction of arrow 610 and arrow 612 in a sweeping motion. The sweeping motion may be from about −180 degrees to about 180 degrees (or any subrange therebetween) of movement about the instrument roll axis 604. The sweeping motion provides a natural wrist turning motion for the user. That is, the instrument roll axis 604 of the system is configured to be substantially aligned with an anatomically natural arm movement of the user. In other embodiments, the instrument roll axis 604 of the system is configured to be offset relative to an anatomically natural arm movement of the user. In some embodiments, the rotatable interface at the handle portion 606 and base portion 608 may be sensorized, for example, with a potentiometer or encoder. As such, the rotational position of the handle portion 606 about the instrument roll axis 604 and with respect to the base portion 608 may be measured and transmitted to the control system 114 to translate the input and carry out movements to control the rotational orientation of the end-effector 124 about the roll axis 405 (illustrated in FIG. 4). The rotational orientation of the end-effector 124 about the roll axis 405 may be proportional to the rotational orientation of the handle portion 606 about the instrument roll axis 604. The change in rotational orientation of the end-effector 124, due to a measured change in the rotational orientation of the handle portion 606 about the instrument roll axis 604, may be made by the rotation of the elongate body 204 illustrated in FIG. 2.

FIG. 7 is an enlarged view of an example control 700 configurable with any of the control systems described herein. The control 700 includes the second interface (e.g., a joystick 314) and a potentiometer interface 702. The second interface (e.g., joystick 314) may be manipulated by a user to provide input to the potentiometer interface 702 to control an orientation of an end-effector of a medical device installed in a control system associated with control 700. The potentiometer interface 702 may work with control system 114 to translate the input and carry out movements to control the orientation of the end-effector (e.g., end-effector 124 illustrated in FIG. 3) with multiple degrees of freedom of the end-effector about a wrist joint of the end-effector. The potentiometer interface 702 may include a sensorized ball joint. The sensorized ball joint may allow the joystick 314 to pitch about the pitch axis 703 and to yaw about the yaw axis 701. The sensorized ball joint may use a plurality of potentiometers or encoders to measure the position of, for example, a joystick 314 with respect to pitch axis 703, yaw axis 701, and/or roll axis 705. The measured position of the joystick 314 may be transmitted to the control system 114 to translate the input and carry out control movements for the orientation of the end-effector.

In operation, the user may orient the end-effector 124 about pitch axis 403, yaw axis 401, and/or roll axis 405 (illustrated in FIG. 4) using a thumb and a finger, one or more fingers, or a plurality of fingers to manipulate the second interface (e.g., joystick 314) about the pitch axis 703, yaw axis 701, and/or roll axis 705 (illustrated in FIG. 7). In some implementations, the joystick 314 represents the second interface of control system 114. The joystick 314 may be engageable by a first set of fingers of the user. For example, the user may place a thumb and at least one finger on the second interface (e.g., a joystick 314) to move the joystick about the pitch axis 703 and yaw axis 701, which in turn actuate the end-effector 124 (illustrated in FIG. 4) about the pitch axis 403 and yaw axis 401, respectively. In addition, the user may use the same thumb and at least one finger to rotate, twist or spin the second interface (e.g., a joystick 314) in a clockwise or counterclockwise direction about the end-effector roll axis 705, as illustrated by arrow 704 and arrow 706. The rotation of the joystick 314, measured by the potentiometer interface 702 and received by the control system 114, may result in the end-effector rotating in the same direction.

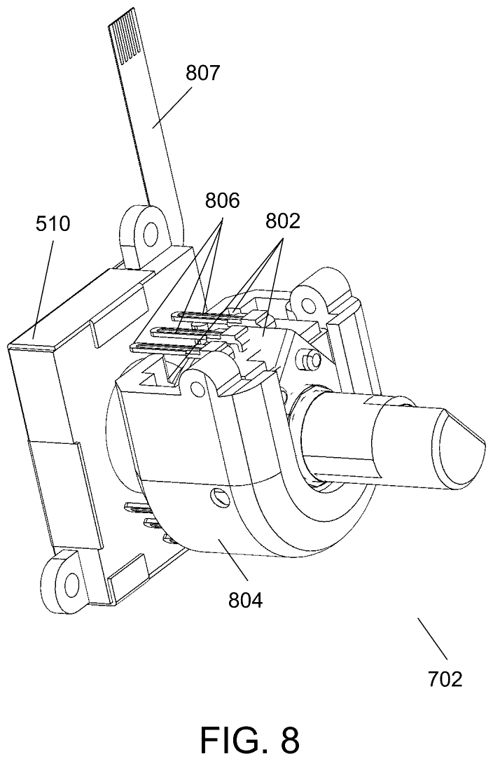

FIG. 8 is an internal perspective view of components configured to actuate the control systems described herein. In particular, the components illustrated here represent the potentiometer interface 702 for the joystick mechanisms described herein. The potentiometer interface 702 includes the switch 510, and a plurality of potentiometers 802, and a potentiometer housing 804. The switch 510 is configured to interface via a lead 807 with one or more circuit boards that have one or more connectors or cables connected to control housing 508 (illustrated in FIG. 5A). In operation, the plurality of potentiometers 802 (or alternatively, encoders) are positioned to measure the orientation of the second interface (e.g., joystick 314). For example, a first potentiometer may be coupled to and measure the position of the joint (e.g., a swivel joint) that the joystick 314 rotates about the roll axis 705 (illustrated in FIG. 7) on, and a second and third potentiometer may be coupled to a ball joint to measure the pitch of the joystick 314 about the pitch axis 703 and the yaw of the joystick 314 about the yaw axis 701. Information from the potentiometers and/or other sensors associated with the joystick may be continuously fed into a suitable controller (e.g., microcontroller, processor, etc.) in the form of suitable sensor signals. The microcontroller can then calculate a desired end-effector wrist orientation based on the orientation of the joystick. The microcontroller may generate corresponding controller output signals and can then send the commands/signals to one or more motors associated with the control system 100. The motors may each have a motor encoder, signals from which can optionally be fed back to the microcontroller via the motor controller. The motors can then actuate the medical device and/or end-effector 124 to change the end-effector orientation based on the outputs of the potentiometer 802.

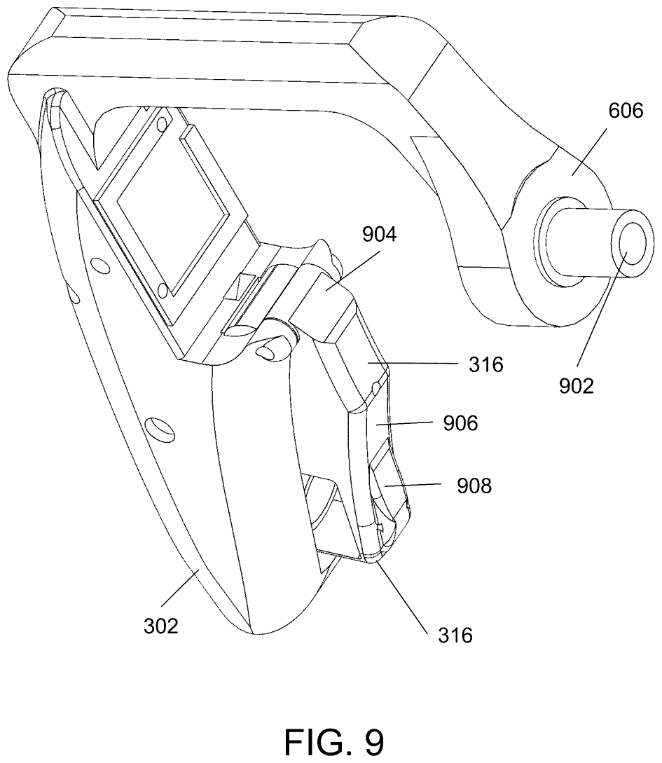

FIG. 9 is a perspective view of an example control 316 configurable with the control systems described herein. The handle 302 is rotatably coupled to the base portion 608 (illustrated in FIG. 6) via a pivot point 902. In this example, the third interface (e.g., compressible control 316) is installed in the handle 302. The third interface (e.g., compressible control 316) includes at least grip locations for two or more fingers. The grip locations are illustrated as a first grip location 904, a second grip location 906, and a third grip location 908.

In operation, the user may operate the third interface (e.g., compressible control 316) by placing a hand around handle 302 and placing a first, a second and/or a third finger on any of the grip locations 904, 906, 908 or intermediate positions therebetween. Once one or more fingers are placed on the third interface (e.g., compressible control 316), the user may squeeze the third interface to trigger at least one function of an end-effector (e.g., end-effector 124 illustrated in FIG. 4) associated with a medical device (not shown) installed in a portion of the control system associated with the handle 302. In some implementations, the user may grasp the handle by placing a palm on the handle 302 and placing a thumb and at least one finger on the joystick (not shown) while placing the remaining fingers on the control 316 at or near grip locations 904, 906, 908. In some implementations, the finger and thumb may be switched from the joystick 314 (illustrated in FIG. 4) to the third interface (e.g., compressible control 316) to engage the third interface to trigger at least one function of the end-effector. In some implementations, the finger that was on the joystick may be the finger used to select (or squeeze) the third interface (e.g., compressible control).

In some implementations, the grip locations 904, 906, 908 may each be associated with a different function for the end-effector. Selecting each different function may cause the end-effector to perform a different function. In some implementations, the user may use the joystick (e.g., the second interface of handle 302) in combination with the third interface (e.g., compressible control 316) to move the medical device in up to four degrees of freedom of movement relative to the base member pivotally connected to the handle 302 via pivot point 902.

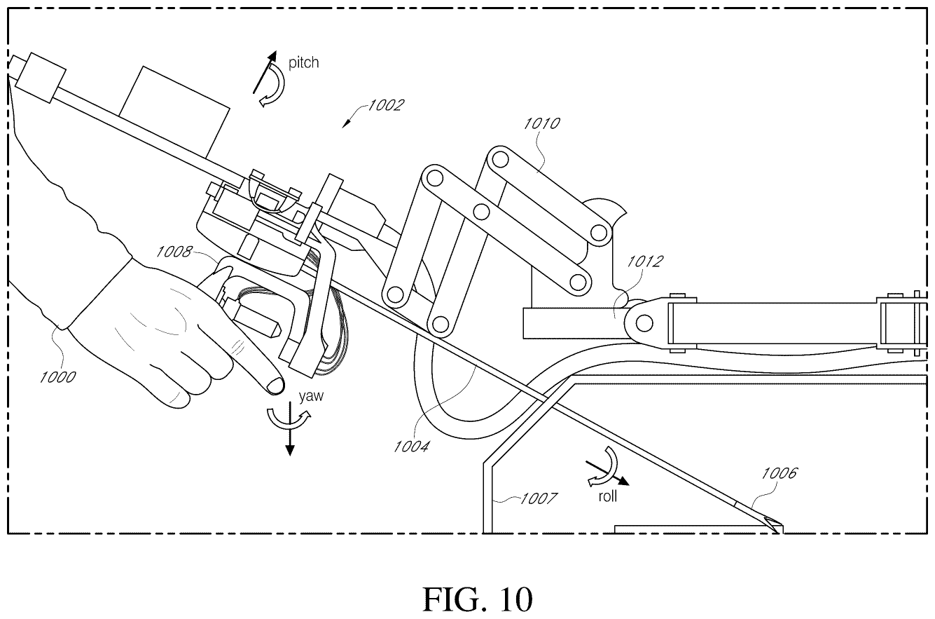

FIG. 10 is a side view of a user 1000 engaging with an example control system 1002 with an installed medical device 1004 that has an end-effector 1006. The user 1000 is illustrated gripping a handle 1008 of the control system 1002 with at least three fingers. In some implementations, the user may grip the handle 1008 with one or more fingers and/or a thumb.

The user may use any grip on handle 1008 to move the medical device 1004 in space to position the device 1004 by maneuvering the first interface (e.g., the handle 1008) using one or both of a forearm and a wrist to cause movement of the medical device along and about both the pitch and yaw axes, using the coupled stabilizing apparatus 1010 (e.g., using a parallelogram movement mechanism) and a remote base 1012 (sometimes also configured as a revolute joint) as a counterbalancing mechanism. In addition, the user may maneuver the handle 1008 using one or both of a forearm and a wrist to cause movement of the medical device 1004 about a roll axis 1007. In this example, the forearm or wrist align with an offset of the roll axis 1007 of the medical device 1004 (illustrated in FIG. 3).

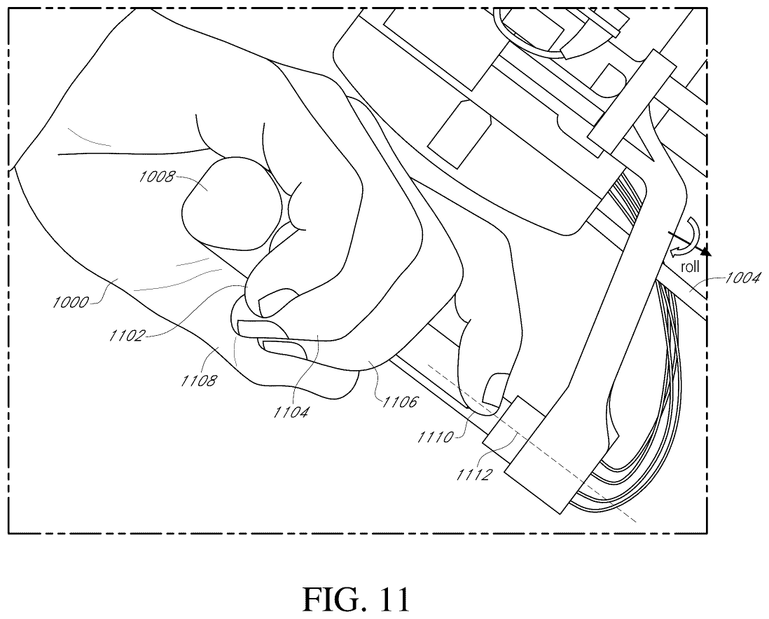

FIG. 11 is a perspective view of a user engaging with at least one control of the example control system of FIG. 10. In this example, the user 1000 is illustrated grasping the handle 1008 with a palm and a finger 1102, a finger 1104, a finger 1106, and a thumb 1108. A finger 1110 may be floating or placed on another portion of the handle 1008, for example an interface, such as a barrel or a joystick. The user may use fewer fingers to grasp handle 1008.

The user may roll the handle 1008 clockwise and counterclockwise about a centerline axis 1112. The centerline axis 1112 is parallel to the instrument roll axis associated with the medical device 1004. The movement of the handle 1008 may be a sweeping motion from about 165 degrees to about 180 degrees of movement about the roll axis. The sweeping motion provides a natural wrist turning motion for the user. That is, the roll axis of system handle 1008 about centerline axis 1112 is configured to be aligned with an anatomically natural arm movement of the user.

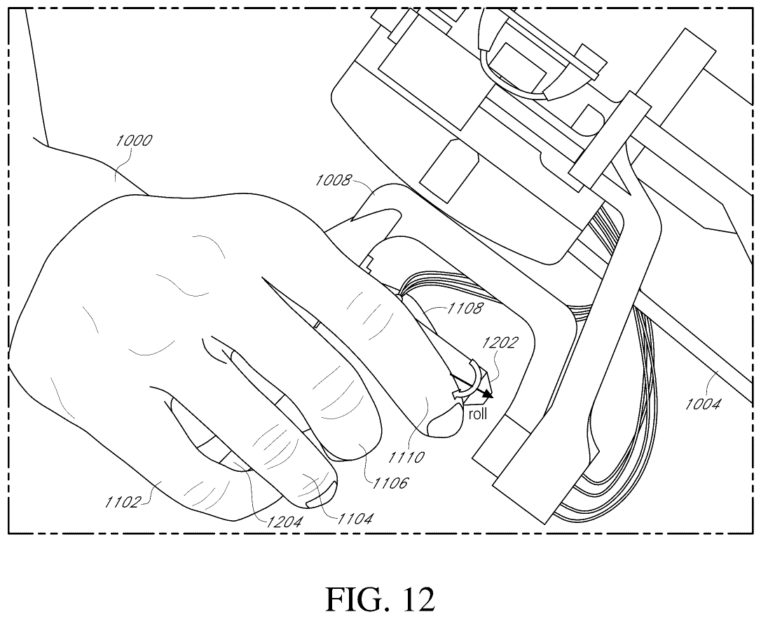

FIG. 12 is a perspective view of a user engaging with at least one control of the example control system of FIG. 10. In particular, the user 1000 has grasped the handle 1008 with a finger 1102, a finger 1104, and a finger 1106 while grasping a joystick control 1202 with thumb 1108 and finger 1110. The user may be ready to engage with the joystick control 1202 to adjust an orientation of an end-effector (not shown) of installed medical device 1004.

In some implementations, the user may engage with the joystick control 1202 using the thumb 1108 and at least one finger (e.g., finger 1110) to twist or spin the joystick control 1202 about the roll axis (illustrated in FIG. 7). This movement may cause a roll (e.g., turn, spin, etc.) of an end-effector (not shown) of the installed medical device 1004. The movement may include turning (e.g., twisting, spinning, etc.) the joystick control 1202 in a clockwise or counterclockwise direction to achieve corresponding clockwise or counterclockwise movement of the end-effector. In some implementations, the user 1000 may also engage with a compressible control 1204 using finger 1102, finger 1104, finger 1106, and/or finger 1110.

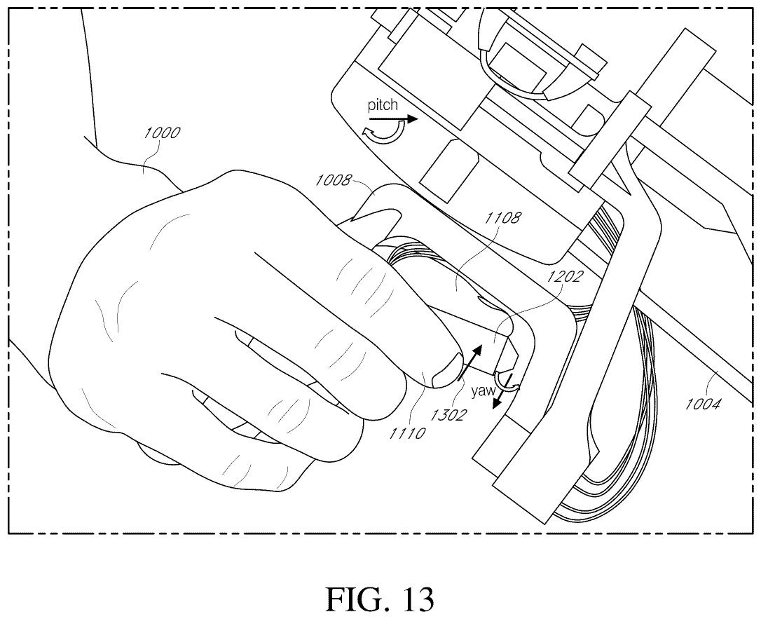

FIG. 13 is a perspective view of a user engaging with at least one control of the example control system of FIG. 10. In this example, the user 1000 has grasped the handle 1008 with one or more fingers while grasping the joystick control 1202 with thumb 1108 and finger 1110. The user has moved the joystick control 1202 in the direction of arrow 1302 to cause movement of an end-effector (not shown) of installed medical device 1004. The movement may articulate the end-effector about the pitch axis. Other movements of joystick control 1202 may include moving the joystick control 1202 about the roll and yaw axes and any angle in between, which in turn actuates an end-effector (not shown) about the pitch and yaw axes, respectively.

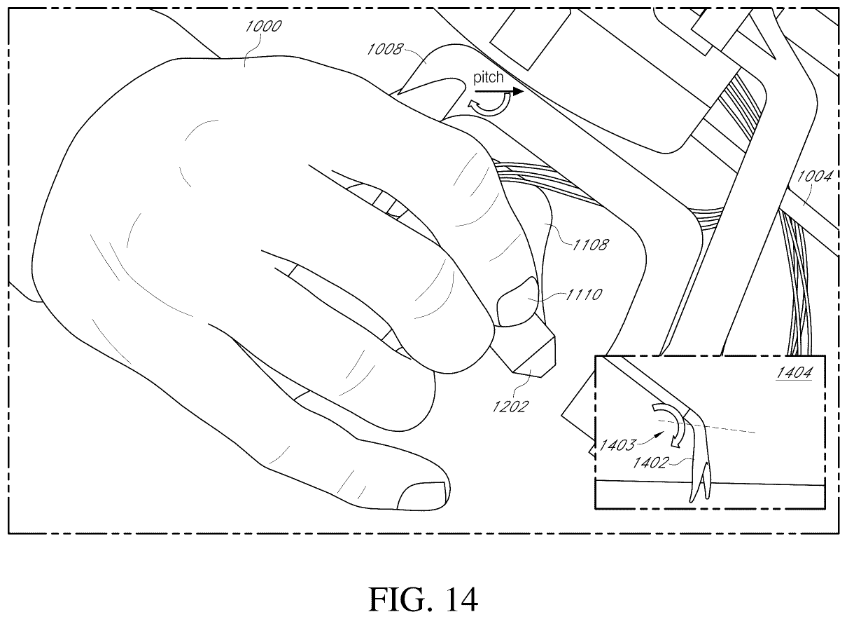

FIG. 14 is a perspective view of a user engaging with at least one control of the example control system of FIG. 10. In this example, the user has a pencil grip of the joystick control 1202 using the thumb 1108 and the finger 1110. The user may maneuver joystick control 1202 at an angle to cause an end-effector 1402 (illustrated in exploded view 1404) of installed medical device 1004 to change orientation. For example, the user has moved the joystick about a pitch axis 1403 (i.e., in a downward direction), which results in the end-effector 124 having an orientation that is downward with respect to the pitch axis 1403. Corresponding movements may also be made in the yaw axis. In some implementations, the user 1000 may use the thumb 1108 to move the joystick control 1202 without engaging fingers on the joystick control 1202.

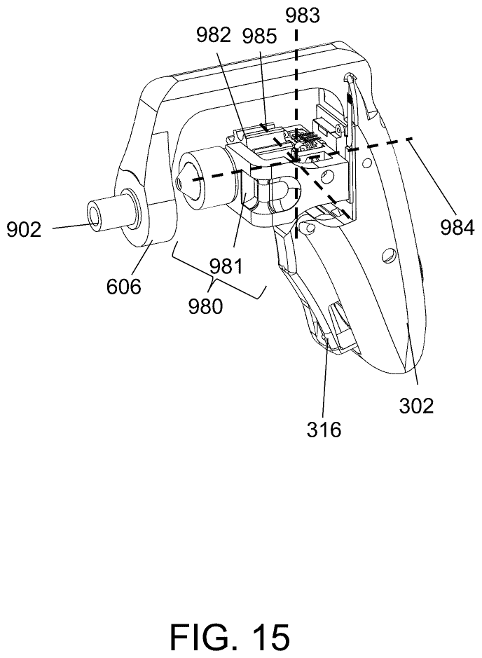

FIGS. 15-18 show various embodiments of control systems that may be used in combination with any of the control systems described above. Further, any of the control systems of FIGS. 15-18 may be used with any of the structures or components described in connection with FIGS. 1-2. FIG. 15 illustrates a perspective side view of an exemplary control system. The control system illustrated in FIG. 15 may represent the control system 114 (illustrated in FIG. 5A). The handle portion 606 may be rotatably coupled to the base portion 608 similar to the base portion shown in FIG. 6. The rotatable interface of the handle portion 606 and the base portion 608 may include a potentiometer (or encoder) to measure the roll position of the handle portion 606 with respect to the base portion 608 about the instrument roll axis 604 (illustrated in FIG. 6). A processor communicatively coupled to the control system may receive the measured position of the handle portion 606 and rotate the elongate body 204 of the medical instrument 118 about the roll axis 127 to a corresponding position. The exemplary control system of FIG. 15 includes a first interface (e.g., a handle 302 as described for FIGS. 2, 4, 5A, 6 and 9), a second interface, and a third interface. The second interface control (illustrated here as a joystick 980) that includes the pitch and yaw control capabilities of the joystick 314 described at least for example in FIGS. 3, 4, 5A, 6 and 7. For example, the second interface (e.g., joystick 980) is manipulatable about a pitch axis 985 and a yaw axis 983 to articulate the end-effector 124 about the pitch axis 403, the yaw axis 401 (illustrated in FIG. 4). The second interface control (e.g., joystick 980) may also include a dial or wheel 982. The wheel 982, depicted as being ergonomically positioned for thumb and/or finger actuation, may include a potentiometer (or encoder), which measures a roll orientation about the roll axis 984. The measured roll orientation may be received by the control system to roll the end-effector in the same direction. For example, if the wheel 982 is rolled to the left, the end-effector 124 would be rolled to the left about the roll axis 405 (illustrated in FIG. 4). Further, the end-effector 124 may be rolled while being pitched and/or yawed. As such, a rolled end-effector actively being pitched and/or yawed would roll about a roll axis offset by the same amount of pitch and/or yaw as the manipulated end-effector. In some embodiments, the second interface (e.g., joystick 980) may include a groove 981, indentation, or the like for positioning a finger or one or more fingers for gripping, manipulating, or otherwise interacting with the second interface (e.g., joystick 980). The control system of FIG. 15 may also include third interface control described, at least for example, for the compressible control 316 of FIGS. 3, 4, 5A, 6 and 9. For example, actuating the third interface (e.g., compressible control 316) may provide an electromechanical actuation of one or more functions of an end-effector of the medical device. The end-effector may have a plurality of predefined functions and such functions may be triggered by a user interacting with the third interface (e.g., compressible control 316). Example functions include, but are not limited to, closing of mechanical jaws, administration of electrical energy, administration of cryotherapy, cutting motions, grasping motions, a material removal function (e.g., cutting, drilling, deburring, etc.), an irrigation function, a suction function, a stapling or adhesive function, etc.

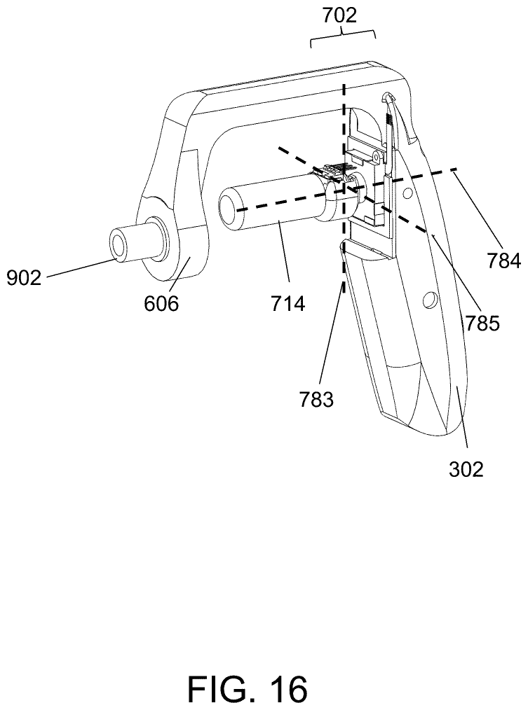

FIG. 16 illustrates a perspective side view of another embodiment of a control system. The control system illustrated in FIG. 16 may represent the control system 114 (illustrated in FIG. 5A). The handle portion 606 may be rotatably coupled to the base portion 608 similar to the base portion in FIG. 6. The rotatable interface of the handle portion 606 and the base portion 608 may include a potentiometer (or encoder) to measure the roll position of the handle portion 606 with respect to the base portion 608 about the instrument roll axis 604 (illustrated in FIG. 6). A processor communicatively coupled to the control system may receive the measured position of the handle portion 606 and rotate the elongate body 204 of the medical instrument 118 about the roll axis 127 to a corresponding position. The illustrated control system includes a first interface (e.g., a handle 302 as described at least for example in FIGS. 2, 4, 5A, 6 and 9), and a second interface (illustrated here as a compressible joystick 714) that includes the capabilities of the joystick 314 described at least for example in FIGS. 3, 4, 5A, 6 and 7. For example, the second interface (e.g., the joystick 714) is manipulatable about a pitch axis 785, a yaw axis 783 and the roll axis 784 to articulate the end-effector 124 about the pitch axis 403, the yaw axis 401, and the roll axis 405 (illustrated in FIG. 4). As such, the joystick 714 may include a potentiometer interface 702 as described at least for example in FIGS. 7-8. The compressible joystick 714 may also include third interface capabilities, such that actuation of an end-effector may be performed with the compressible joystick 714 (e.g., at least some of the control described for the compressible control 316 of FIGS. 3, 4, 5A, 6 and 9). The compressible joystick 714 may include one or more pressure sensors, and, as such, may measure the amount of pressure applied onto by one or more fingers and/or a thumb. The measured pressure applied may be received by the control system, which may generate control outputs actuating the end-effector. For example, if the end-effector 124 is a clasp type as illustrated in FIG. 4, the amount of pressure measured at the compressible joystick 714 may indicate what percent open the clasp end-effector 124 may be. For example, if the pressure is 50% of the pressure threshold for a fully closed clasp-type end-effector 124, the end-effector 124 would be 50% open. Further, if the pressure measured is 100% of the pressure threshold for a fully closed clasp-type end-effector 124, the end-effector 124 would be 100% closed. If there is zero measured pressure, the end-effector 124 would be 100% open. Any other intermediate position is also contemplated.

FIG. 17 illustrates a perspective side view of another variation of a control system. The control system illustrated in FIG. 17 may represent the control system 114 (illustrated in FIG. 5A). The handle portion 606 may be rotatably coupled to the base portion 608 illustrated in FIG. 6. The rotatable interface of the handle portion 606 and the base portion 608 may include a potentiometer (or encoder) to measure the roll position of the handle portion 606 with respect to the base portion 608 about the instrument roll axis 604 (illustrated in FIG. 6). A processor communicatively coupled to the control system may receive the measured position of the handle portion 606 and rotate the elongate body 204 of the medical instrument 118 about the roll axis 127 to a corresponding position. The illustrated control system includes a first interface (e.g., a handle 302 as described for FIGS. 2, 4, 5A, 6 and 9), a second interface, and a third interface. The second interface control (as described at least for example as a joystick 314 of FIGS. 3, 4, 5A, 6, and 7) may be performed by an exterior joystick 952 and a wheel 954. The exterior joystick 952 may include one or more potentiometers (or alternatively, encoders), which measure the pitch of the exterior joystick 952 about the pitch axis 956 and measure the roll of the exterior joystick 952 about the roll axis 958. The measured pitch position of the exterior joystick 952 may be received by the control system to move the end-effector in the same pitch direction. For example, if the exterior joystick 952 is pitched forward (away from the user), the end-effector 124 would be pitched downward about the pitch axis 403 (illustrated in FIG. 4). The measured roll position of the exterior joystick 952 may be received by the control system to move the end-effector in a corresponding yaw rotation. For example, if the exterior joystick 952 is rolled to the left, the end-effector may yaw to the left in a corresponding manner. The wheel 954, depicted as being ergonomically positioned for thumb actuation, may include a potentiometer (or encoder), which measures it's roll orientation about the roll axis 960. The measured roll orientation may be received by the control system to roll the end-effector in the same direction. For example, if the wheel 954 (illustrated as positioned for thumb use but could be positioned for use by any other finger) is rolled to the left, the end-effector 124 would be rolled to the left about the roll axis 405 (illustrated in FIG. 4). The third interface control (e.g., at least some of the control is described for example with respect to the compressible control 316 of FIGS. 3, 4, 5A, 6 and 9) of the control system of FIG. 17 may be performed with a compressible control 950. Unlike the described compressible control 316 of FIGS. 3, 4, 5A, 6 and 9, in which it is located in the second portion 309 of the handle 302, the compressible control 950 of the embodiment of FIG. 17 is located in the first portion 305 of the handle 302. Positioning the compressible control 950 in the first portion 305 of the handle 302 may be suitable for use with the index finger and/or other finger of a user.

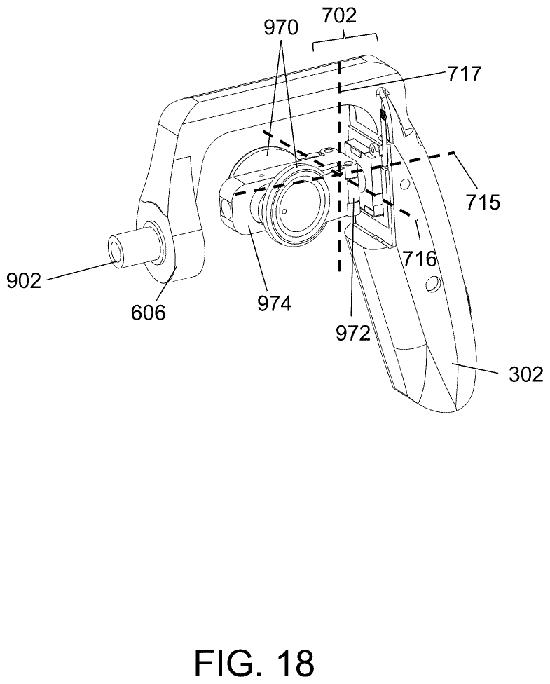

FIG. 18 illustrates a perspective side view of an exemplary control system. The control system illustrated in FIG. 18 may represent the control system 114 (illustrated in FIG. 5A). The handle portion 606 may be rotatably coupled to the base portion 608 illustrated in FIG. 6. The rotatable interface of the handle portion 606 and the base portion 608 may include a potentiometer (or encoder) to measure the roll position of the handle portion 606 with respect to the base portion 608 about the instrument roll axis 604 (illustrated in FIG. 6). A processor communicatively coupled to the control system may receive the measured position of the handle portion 606 and rotate the elongate body 204 of the medical instrument 118 about the roll axis 127 to a corresponding position. The exemplary control system includes a first interface (e.g., a handle 302 as described for FIGS. 2, 4, 5A, 6 and 9), a second interface, and a third interface. The second interface control (e.g., as described at least for example with respect to the joystick 314 of FIGS. 3, 4, 5A, 6, and 7) may be performed by the joystick 974. For example, the second interface (e.g., the joystick 974) is manipulatable about a pitch axis 716, a yaw axis 717, and the roll axis 715 to articulate the end-effector 124 about the pitch axis 403, the yaw axis 401, and the roll axis 405 (illustrated in FIG. 4). The joystick 974 may include a potentiometer interface 702 as described for FIGS. 7-8. The measured movements of the joystick 974 received by the control system may actuate movements of an end-effector as described for the joystick 314 of FIGS. 3, 4, 5A, 6, 7 and 8. The second interface (e.g., joystick 974) may also include a third interface control (e.g., including at least some of the capabilities described for the compressible control 316 of FIGS. 3, 4, 5A, 6 and 9). As illustrated in FIG. 18, the joystick 974 may include one or more paddles 970 hingedly coupled to the joystick 974. The hinge coupling of each paddle 970 may include a potentiometer (or encoder) to measure the orientation the paddle 970 with respect to the hinge 972 that each paddle 970 is coupled to. A processor communicatively coupled to the control system may receive the measured orientation of one or more paddles 970 and actuate the end-effector accordingly. For example, if the end-effector 124 is a clasp-type as illustrated in FIG. 4, the control system may move the jaws of the end-effector 124 to position corresponding to the measured position of the paddles 970. For example, if the paddles 970 are moved to and measured at a position that is 50% of open, the control system will move the end-effector 124 jaws to a point that is 50% of open. Any other intermediate position is also contemplated. The paddles 970 may be biased to, for example, the open position. The paddles 970 may be biased to the open position by, for example, torsional springs. Embodiments with paddles 970 biased to the open position may allow a user to press the paddles toward the close position with, for example, a thumb and an index finger. Release of pressure upon paddles 970 biased towards the open position allows them to return towards or to the open position.

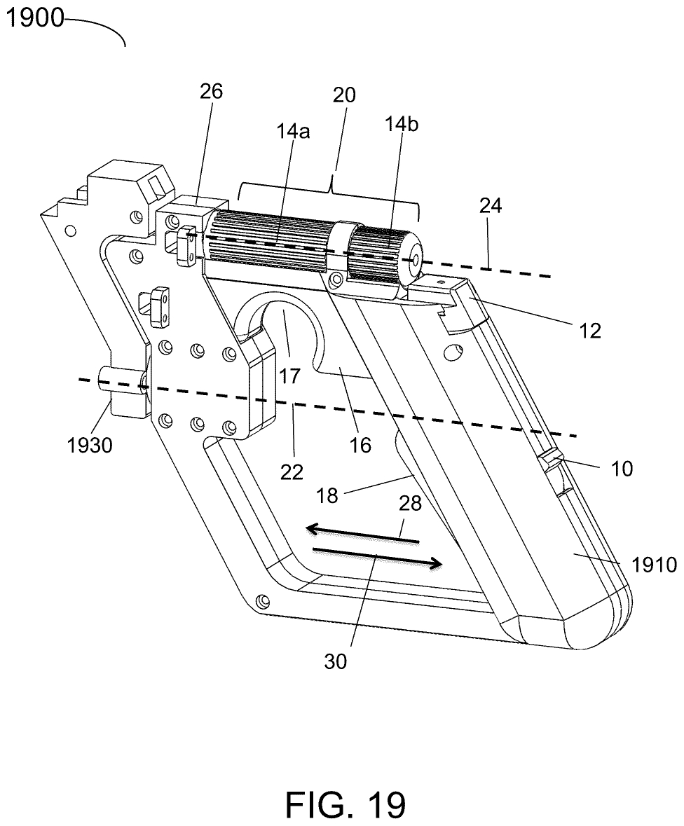

FIG. 19 illustrates a perspective view of an exemplary control system 1900. The control system 1900 may include a handle portion 1910, a mode toggle 10, a trigger release 12, a trigger 16, a roll barrel 20, and an enablement switch 18. The handle portion 1910 may be rotatably coupled to the base portion 1930, similarly described in FIG. 6. The rotatable interface of the handle portion 1910 and the base portion 1930 may include a potentiometer (or encoder) to measure a roll position of the handle portion 1910 with respect to the base portion 1930 about a roll axis 22. A processor may receive the measured position of the handle portion 1910 and rotate an elongate body 204 of the medical instrument 118 (shown in FIG. 2) about the roll axis 22 (e.g., roll axis 127 in FIG. 2) to a corresponding position. The exemplary control system 1900 includes a first interface (e.g., a handle portion 1910 similar to those described for FIGS. 2, 4, 5A, 6 and 9), a second interface (e.g., barrel 20), and a third interface (e.g., trigger 16).

The second interface control may include a roll barrel 20. In some embodiments, the roll barrel 20 includes two portions: a first portion 14a and a second portion 14b. In some embodiments, the roll barrel 20 portions 14a and 14b may be coupled together. Alternatively, in some embodiments, roll barrel 20 may include one portion, two portions, or one or more portions. The roll barrel 20 may be operatively coupled to the control system via a barrel coupling 26. The barrel coupling 26 may include a rolling mechanism (e.g., a roller bearing, etc.) and a position measuring sensor (e.g., a potentiometer, an encoder, etc.) for measuring a roll orientation of the roll barrel 20 about a roll axis 24. The roll barrel 20 may be rotated about a roll axis 24 via a finger and/or a thumb in contact with the first portion 14a and/or the second portion 14b. The rotation of the roll barrel 20 may be measured by the position measuring sensor of the barrel coupling 26 and may be received by the control system. The measured roll orientation may be received by the control system to roll the end-effector in the same direction. For example, if the roll barrel 20 is rolled to the left, the end-effector 124 would be rolled to the left about the roll axis 405 (e.g., see also FIG. 4).

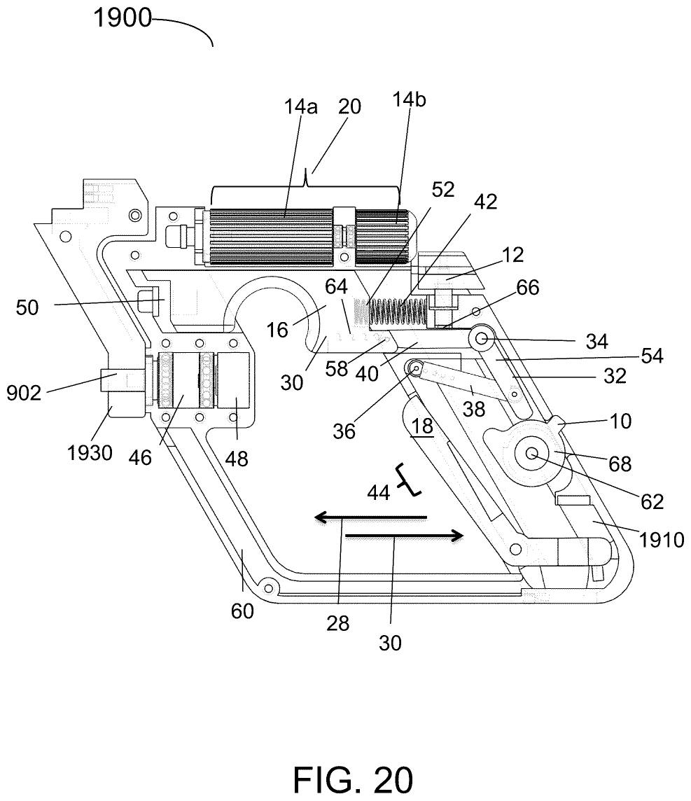

The control system 1900 illustrated in FIG. 19 includes a third interface, for example a trigger 16 (e.g., including at least some of the capabilities described for the compressible control 316 of FIGS. 3, 4, 5A, 6 and 9). The trigger 16 may be positioned appropriately for use by, for example, an index finger within the finger interface 17 (e.g., a concave section, groove, indentation, and the like). The trigger 16 may be biased, for example, in the direction 28, for example towards a base portion 1930 or away from a handle portion 1910). A user may use their finger to pull the trigger 16 back against the bias force (i.e., towards handle portion 1910 or away from base portion 1930) producing at least some of the effects described for the compressible control 316 of FIGS. 3, 4, 5A, 6 and 9. For example, actuating the trigger 16 may provide an electromechanical actuation of one or more functions of an end-effector of the medical device. The end-effector may have a plurality of predefined functions and such functions may be triggered by a user interacting with the trigger 16. Example functions include, but are not limited to, closing of mechanical jaws, administration of electrical energy, administration of cryotherapy, cutting motions, grasping motions, a material removal function (e.g., cutting, drilling, deburring, etc.), an irrigation function, a suction function, a stapling or adhesive function, etc. To achieve the opposite effect of pulling the trigger 16 back in the direction 30, the pressure applied on the trigger 16 may be released or reduced to cause the trigger 16 to move back in the direction 28 due to the bias nature of the trigger 16. Some embodiments may include a trigger 16, which is biased in both directions. Embodiments including a dual-biased trigger 16 could include a balanced trigger 16 position in which the biased component forcing the trigger 16 in direction 28 is inactive or balanced by the biased component forcing the trigger 16 in direction 30. If the biased component forcing the trigger 16 in direction 28 is inactive at the described balanced trigger position, so too would the biased component force the trigger 16 in direction 30. A dual-biased trigger 16 may require user to push the trigger 16 for movement of the trigger 16 in direction 28 and may require the user to pull the trigger 16 for movement in direction 30. Dual-biased triggers, triggers biased in direction 28, and triggers biased in direction 30 may include an internal locking mechanism (illustrated in FIG. 20) which locks the trigger 16 in any position in which a bias force is acting upon the trigger 16 (referred to as trigger-locking). Trigger-locking may be released by the pressing of the trigger release 12. The trigger-release 12 may unlock the internal locking mechanism allowing the biased component to force the trigger 16 to a point that bias forces are balanced or are no longer present. Trigger-locking may be a default operation of the control system or may be entered into as a mode of operation. The mode toggle 10 may be used to switch between one or more modes and trigger-locking mode.

FIG. 20 illustrates various exemplary internal mechanisms of control system 1900. The internal mechanisms may include a ratchet mechanism 54, a rack 30, a first trigger spring 42, a mode selector mechanism 68, a roll mechanism 46, and a damper 48. The roll mechanism 46 (e.g., a roller bearing, bushing, etc.) and damper 48 may allow for damped roll of the control system about the roll axis 22 (shown, e.g., in FIG. 19). Damping the roll of a first interface (handle portion 1910 for gross manual movement) about base portion 1930 allows for smooth and/or precise measured movements. Additionally, the damper 48 may hold the position of the first interface or control system 1900, relative to base portion 1930, when the roll force is removed or when the user removes their hand from the control system. Any of the embodiments (e.g., FIGS. 3-18) described herein can include a similar damper or damper mechanism for the first interface and/or the control system. The roll axis 22 (illustrated in FIG. 19) may approximately align with the wrist and/or forearm roll axis (not shown) of the user, increasing the ergonomics of the user movement. The range of gross roll of the first interface and/or control system 1900 may be limited (e.g., 90 degrees, 180 degrees, 270 degrees, etc.) or may be unlimited (e.g., beyond 360 degrees).

FIG. 20 illustrates an example of the biased component of the trigger 16 described herein. As illustrated, the first trigger spring 42 is positioned on a first side 52 of the trigger 16. The first trigger spring 42 may be a tension spring and act in biasing the trigger in direction 30 (e.g., towards handle portion 1910), or the first trigger spring 42 may be a compression spring and act in biasing the trigger in direction 28 (e.g., towards base portion 1930). A second trigger spring (not shown) may be positioned on a second side 50 of the trigger 16. The second trigger spring (not shown) may be a tension spring and act in biasing the trigger in direction 28, or the second trigger spring may be a compression spring and act in biasing the trigger in direction 30. Some embodiments may include a first trigger spring 42 and a second spring (not shown but described herein) for a dual-biased trigger 16 as described herein.

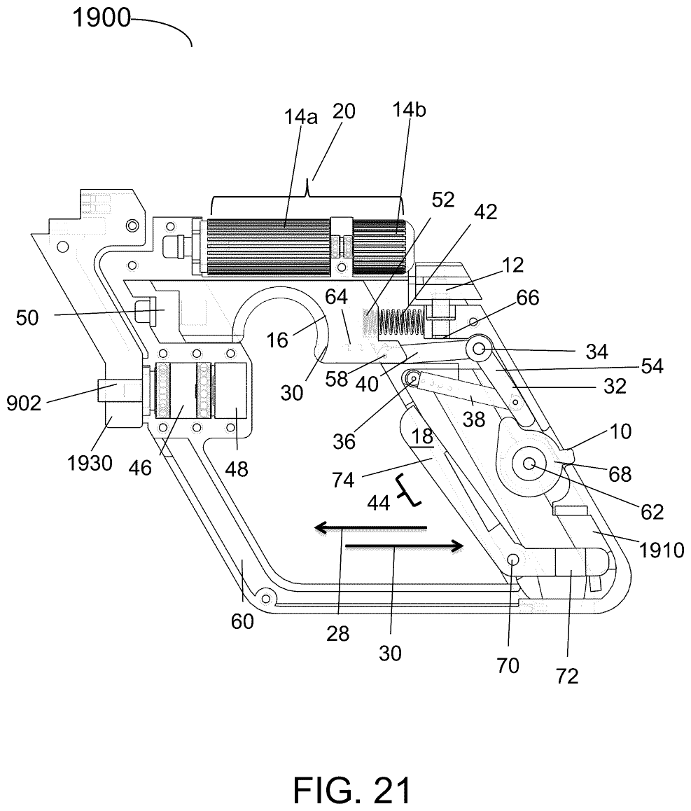

FIG. 20 illustrates an example trigger-locking mechanism. The trigger-locking mechanism of FIG. 20 is a ratchet mechanism, including, a first ratchet element 32, a second ratchet element 38, and a third ratchet element 40. The third ratchet element 40 may include a pall pin 58 that engages with a ratchet rack 30 coupled to the trigger 16 when the control system 1900 is in trigger-locking mode. The first ratchet element 32 may be rigidly coupled to the third ratchet element 40, and, as coupled, may rotate about pivot point 34 braced to the handle housing 60. As such, if the first ratchet element 32 is forced towards the enablement switch 18, a moment (e.g., a clockwise moment with respect to FIGS. 20 and 21) is created upon the third ratchet element 40 forcing the pall pin 58 towards, and into engagement with, the ratchet rack 30. Inversely, if the first ratchet element 32 is forced away from the enablement switch 18, a moment (e.g., a counterclockwise moment with respect to FIGS. 20 and 21) is created upon the third ratchet element 40 forcing the pall pin 58 away from, and out of engagement with, the ratchet rack 30. The second ratchet element 38 may include a bias component (e.g., a tension spring) that creates a contraction force towards the pivot point 36 (which couples the second ratchet element 38 to the handle housing 60) away from the first ratchet element 32. The second ratchet element 38 creates a force pulling the first ratchet element 32 towards the enablement switch 18, creating a moment upon the third ratchet element 40 that engages the pall pin 58 with the ratchet rack 30. As such, the illustrated shape of the ratchet rack 30 teeth allows the pall pin 58 to follow the curvature of the ratchet rack 30 when the trigger 16 is squeezed. At any point that squeeze pressure is reduced or removed, the pall pin 58 will engage with a respective valley 64, locking the trigger 16 in its respective position. The engagement of the ratchet system 54 may be removed by either the trigger release 12 or the mode selector mechanism 68.

Mode selection in some embodiments may be accomplished with a mechanical mode selector as illustrated in FIGS. 20 and 21. Alternatively, mode selection may be a control mode entered into by the control system based on the reception of user inputs by the control system (e.g., pressing mode selection button that generates a signal to the control system) or based on predefined controls for unique medical instruments (e.g., input by a user). Further, some selected modes may include restricted movement (e.g., the control system may not perform certain actions. Some embodiments may include haptic feedback, such as, handle vibrations to indicate to a user different states or modes. The mode selector mechanism 68 illustrated in FIGS. 20 and 21 may include a cam portion which rotates about a pivot point 62. The cam portion of the mode selector mechanism 68 may include a varying radius 44. When the mode selector mechanism 68 is rotated about a pivot point 62 (which is coupled to the handle housing 60) and out of trigger-locking mode by the mode toggle 10 (illustrated in FIG. 21), the radius 44 of mode selector mechanism 68 may increase. Increasing the radius 44 creates contact between the mode selector mechanism 68 and the first ratchet element 32, forcing the first ratchet element 32 away from the enablement switch 18 (i.e., overpowering the bias component of the second ratchet element 38), thus disengaging the ratchet mechanism 54, and entering the control system into trigger-unlocked mode (illustrated in FIG. 21). The trigger release 12 may be used to momentarily disengage the ratchet mechanism 54. If the trigger release 12 is pressed toward the third ratchet element 40, a first portion 66 of the trigger release 12 can press the third ratchet element 40 away from the trigger release 12, disengaging the pall pin 58 from the ratchet rack 30. The trigger release 12 may include a bias component (e.g., a compression spring) which biases the trigger release 12 away from the third ratchet element 40. As such, releasing the trigger release 12 allows the pall pin 58 to return to engagement for the next trigger 16 actuation. Although trigger-locking is described in specific with the control system embodiment of FIGS. 19-21, trigger-locking and associated mechanisms (e.g., ratchet mechanism 54) may be used, for example, in the control systems of FIGS. 3, 4, 5A, 5B, 6, 9, 15, 17, and 22. Additionally, any embodiment utilizing trigger-locking may benefit from the switching (e.g., with mode toggle 10) between at least trigger-locking and trigger-unlocked modes.

Trigger-locking, as described herein, is mechanically performed, but roboticized trigger-locking has been contemplated, for example, autonomous braking for retaining the position of control element (e.g., trigger 16, trigger 216, compressible control 316, etc.). Robotic surgical systems (or any instance where an instrument may have multiple functions or multiple instrument types may be used with the same handle) may benefit from trigger-locking, especially those in which trigger position corresponds to grasp pressure, for example, end-effectors such as needle drivers (at least in some instances) may benefit from locking the grasp pressure onto a needle during suturing and easily unlocking to relieve grasp pressure. Another example of a system that may benefit from trigger-locking may be plain graspers. Plain graspers may be used to engage a material (e.g., tissue during tissue retraction) and it may be beneficial to lock the engagement pressure of the graspers. Further, some robotic surgical systems may benefit from a trigger-unlocked mode, for example, a system utilizing scissors or shears may increase performance with trigger-unlocked mode. In addition, robotic surgical systems in which end-effector actuation has increased performance with rapid activation and rapid deactivation may benefit from trigger-unlocked mode. For example, a bipolar electrocautery end-effector may benefit from trigger-unlocked mode and the capability of intermittent controlled energy application.

FIG. 21 illustrates an example of an enablement switch 18 of a control system 1900. The enablement switch 18 may include a first portion 74, a second portion 72, and a pivot 70. The first portion 74 of the enablement switch 18 may be contacted by a user grasping the handle 302. Grasping the handle 302 may force the enablement switch 18 into, or at least partially, into the handle housing 60, and cause a moment upon the second portion 72 about the pivot 70. The actuation of the second portion 72 may activate a normally-open contact to close or may activate a normally-closed contact to open. The control system may receive a signal from the normally-open contact or may lose a signal across the normally-closed contact, and produce control signals to enable use of controls (i.e., unlock braking, or enter active mode). Alternatively, the enablement switch 18 may include a magnet, while a portion of the handle housing near the magnet of the enablement switch may include a hall effect sensor (or vice-versa). The control system may receive a signal from the hall effect sensor measuring position of the enablement switch 18 based on the magnetic field of the magnet. If the measured magnetic field of the magnet is beyond or within a predefined threshold, the control system may produce control signals to enable use of controls (i.e., unlock braking, or enter active mode). The enablement switch 18 may include a bias component (e.g., a compression spring) which holds the enablement switch 18 in the unactuated position. Alternatively, the enablement switch 18 may include a magnetic latch which biases the enablement switch 18 in an unactuated position.

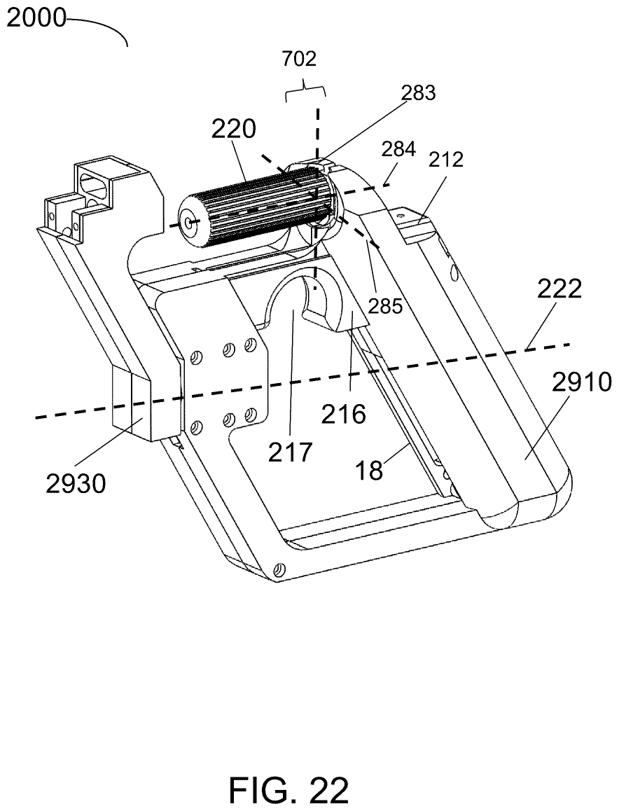

FIG. 22 illustrates a perspective view of an exemplary control system 2000. The control system 2000 may include a handle portion 2910, a mode toggle 10 (shown in FIGS. 19, 20 and 21), a trigger release 212, a trigger 216, a barrel joystick 220, and an enablement switch 218. The handle portion 2910 may be rotatably coupled to the base portion 2930, similarly described in FIG. 6. The rotatable interface of the handle portion 2910 and the base portion 2930 may include a potentiometer (or encoder) to measure a roll position of the handle portion 1910 with respect to the base portion 2930 about a roll axis 222. A processor communicatively coupled to the control system 2000 may receive the measured position of the handle portion 2910 and rotate an elongate body 204 of the medical instrument 118 (shown in FIG. 2) about the roll axis 222 (e.g., roll axis 127 in FIG. 2) to a corresponding position. The exemplary control system 2000 includes a first interface (e.g., a handle portion 2910 similar to those described for FIGS. 2, 4, 5A, 6 and 9), a second interface (e.g., barrel joystick 220), and a third interface (e.g., trigger 216).

FIG. 22 illustrates the second interface control which may include the barrel joystick 220. The second interface (illustrated here as the barrel joystick 220) that includes the capabilities of the joystick 314 described at least for example in FIGS. 3, 4, 5A, 6 and 7. The barrel joystick 220 may be positioned for manipulation by one or more fingers and/or a thumb. For example, the second interface (e.g., the joystick 220) is manipulatable about a pitch axis 285, a yaw axis 283, and the roll axis 284 to articulate the end-effector 124 about the pitch axis 403, the yaw axis 401, and the roll axis 405 (illustrated in FIG. 4). As such, the joystick 714 may include a potentiometer interface 702 as described at least for example in FIGS. 7-8.

The control system 2000 illustrated in FIG. 22 includes a third interface, for example a trigger 216 (e.g., including at least some of the capabilities described for the compressible control 316 of FIGS. 3, 4, 5A, 6 and 9). In some embodiments, the trigger 216 may be substantially similar to the trigger 16 of FIGS. 19, 20 and 21, and may include trigger-locking capabilities described in FIGS. 20 and 21. The trigger 216 may be positioned appropriately for use by, for example, an index finger within the finger interface 217 (e.g., a concave section, groove, indentation, and the like). The trigger 216 may be used to produce at least some of the effects described for the compressible control 316 of FIGS. 3, 4, 5A, 6 and 9. For example, actuating the trigger 216 may provide an electromechanical actuation of one or more functions of an end-effector of the medical device. The end-effector may have a plurality of predefined functions and such functions may be triggered by a user interacting with the trigger 16. Example functions include, but are not limited to, closing of mechanical jaws, administration of electrical energy, administration of cryotherapy, cutting motions, grasping motions, a material removal function (e.g., cutting, drilling, deburring, etc.), an irrigation function, a suction function, a stapling or adhesive function, etc.

Although robotic surgical system embodiments described herein are in the singular form, it is contemplated that two or more robotic surgical systems may be mounted to the patient support apparatus 102 (illustrated in FIG. 1) or proximally to a surgical site. The ergonomic and intuitive features described herein may be used to operate a robotic surgical system and one or more companion systems in collaboration, iteratively, sequentially, or concomitantly during, for example, a minimally invasive surgery.

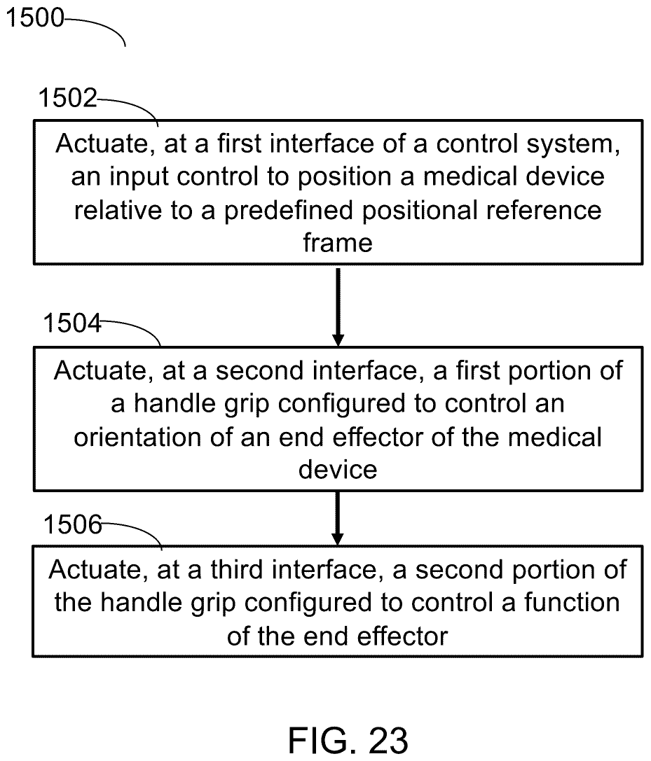

FIG. 23 is an example flow diagram of a method 1500 of performing a surgical procedure with the control systems described herein. The method of performing the surgical procedure may be performed with the robotic surgical system 100 utilizing medical instrument 118 having end-effector 124. The method 1500 may represent a method of treatment, a method of performing a minimally invasive surgery, a method of performing a surgical procedure, or the like.

The method 1500 may be carried out using a robotic surgical system 100 that includes a number of robotic-assisted components as well as manually movable components. Such a system may include at least a handle 103, 302 (with a handle grip 304) coupled to a control system 114 that may be manipulated by a forearm and/or wrist, and/or one or more fingers of a user (e.g., a surgeon). For example, any of the systems, components, interfaces, or handle assemblies of FIGS. 1-18 may be used in the method 1500 of FIG. 23. The control system 114 may include a number of user interfaces for actuating medical devices and end-effectors of such devices. For example, a first interface may be provided as a handle/handle grip (e.g., handle 302, handle grip 304) while a second interface may be provided as a joystick control (e.g., a joystick), and a third interface may be provided as a compressible control (e.g., compressible control 316). A user may engage with the interfaces described herein using one or more portions of a hand (e.g., a palm, a finger, a thumb, a knuckle, etc.) and/or a forearm or wrist.

At block 1502, the method 1500 includes actuating, at a first interface of a control system, an input control to position a medical device relative to a selected positional reference frame. For example, an input received at the first interface (e.g., the handle 302) may cause movement of the medical instrument 118 to be positioned in space relative to a positional reference frame associated with the handle 302 or relative to a remote center of motion of the system. In some implementations, the positional reference frame may be defined by a horizontal or vertical centerline associated with the handle 302, the medical instrument 118, and the like. In some implementations, the positional reference frame may be defined by a portion of the control system 114 that is coupled to the handle 302. In some implementations, the positional reference frame may be defined by a remote component that is affixed to an arm when the arm is also affixed to the control system 114 and/or handle 302. In some implementations, the positional reference frame may be defined relative to a remote center of motion, for example at an incision or surgical access site of the patient.

In some implementations, actuating the input control (e.g., the handle 302 at the first interface) includes maneuvering the first interface (e.g., the handle) using one or both of a forearm and a wrist to cause movement of the medical instrument 118 about an instrument roll axis associated with the selected positional reference frame. For example, the actuation may be a twisting motion of the handle 302 about a pivot point where the twisting or pivoting motion includes the user grasping the handle 302 and twisting the handle clockwise or counterclockwise about the centerline of the roll axis associated with the handle (e.g., axis 307. In this example, the forearm or wrist of a user may be offset from the roll axis associated with the medical instrument 118.

At block 1504, the method 1500 includes actuating, at a second interface, a first portion of a handle grip configured to control an orientation of an end-effector of the medical device. For example, an input received at the second interface (e.g., any of the second interfaces of FIGS. 3-18) may cause movement to change an orientation of the end-effector 124 of the medical instrument 118. The second interface (e.g., any of the second interfaces of FIGS. 3-18) may include one or more sensors in a sensor assembly. The sensor assembly may monitor input detected at the second interface 314 (e.g., joystick, wheel, barrel, etc.) and provide detected input to one or more controllers, electronics, and the like, to carry out end-effector orientation changes according to the detected input.

In some implementations, actuating the first portion at the second interface includes a user gripping the first portion (e.g., the second interface 314) of the handle grip 304 with a thumb and a finger. That is, to actuate the second interface, a user may move the second interface 314 (e.g., a joystick) by gripping the joystick of the handle grip 304 with a thumb and a finger. Movements performed at the second interface 314 (e.g., a joystick) may cause a change in orientation of the end-effector 124 about an end-effector pitch axis, an end-effector yaw axis, and/or an end-effector roll axis (illustrated FIG. 4).

At block 1506, the method 1500 includes actuating, at a third interface, a second portion of the handle grip configured to control a function of the end-effector. For example, an input received at the third interface (e.g., the compressible control 316) may trigger a function of the end-effector 124 of the medical instrument 118, as described herein. In some implementations, actuating the second portion at the third interface comprises gripping the second portion (e.g., lower portion) of the handle grip 304 with one or more fingers of the user. Any of the third interfaces of any of the control systems of FIGS. 3-18 may be used.

In some implementations, the third interface (e.g., the control 316) may be coupled to the sensor assembly to monitor input detected at the third interface and to provide detected input to one or more controllers, electronics, and the like, to carry out end-effector functions according to the detected input. In some implementations, actuation of each of the input control, the first portion, and the second portion is mapped to a separate hand portion or body portion, as described in herein. Such mapping may improve ergonomics of the user and/or allow medical instrument positional control independent of end-effector orientation control.

Although the control systems described herein are described in the context of surgical applications, for example MIS, one of skill in the art will appreciate that the controls, interfaces, and related methods described herein may be used in maintenance, manipulation, assembly, applied robotics, manufacturing, machining, warehouse applications, etc., without departing from the scope of the present disclosure.

The systems described herein may be useable with many different types of medical instruments. As such, the methods of use are to remain substantially the same as to further the intuitive nature of using different types of medical instruments with robotic surgical systems described herein. For example, regardless of what type of medical instrument is loaded into the system, the first interface of the system may be used for gross positioning of the medical instrument, the second interface of the system may be used for robotically assisted movement of the medical instrument, and the third interface of the system may be used for the actuation of the end-effector of the medical instrument. For example, in some embodiments, a method of using any of the control systems described herein includes: loading a first type of medical instrument into a device attachment unit; manipulating the first type of medical instrument with a control system attached to the device attachment unit (e.g., including a first interface, a second interface, and/or a third interface); decoupling or unloading the first type of medical instrument from the device attachment unit; loading a second type of medical instrument into the device attachment unit; and manipulating the second type of medical instrument with the control system.

The systems and methods of the various embodiments and variations thereof can be embodied and/or implemented at least in part as a machine configured to receive a computer-readable medium storing computer-readable instructions. The instructions are preferably executed by computer-executable components preferably integrated with the system and one or more portions of the processor communicatively coupled to an actuating apparatus and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “limit” may include, and is contemplated to include a plurality of limits. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 10%, 5%, 1% or 0.1%. All numerical ranges provided herein are inclusive of the stated start and end numbers. The term “substantially” indicates mostly (i.e., greater than 50%) or essentially all of a device, substance, or composition. For the lengths and widths described herein about may, in some examples, mean plus or minus 10% of the stated value but is not limited to exactly 10% or less in all situations.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments illustrated. This disclosure is intended to cover any and all adaptations or variations of various embodiments. Combinations of the above embodiments, and other embodiments not specifically described herein, will be apparent to those of skill in the art upon reviewing the above description.

### EXAMPLES

Example 1. A control system for a medical device, the control system comprising: a first interface configured to control a position of the medical device, the first interface comprising a handle grip coupled to a base member, the base member being coupled to a device attachment unit configured to couple to the medical device; a second interface installed in a first portion of the handle grip and configured to control an end-effector orientation of an end-effector of the medical device, the second interface being communicatively coupled to a sensor assembly to monitor movement detected at the second interface; and a third interface installed in a second portion of the handle grip and configured to control a function of the end-effector, the third interface being communicatively coupled to the sensor assembly to monitor movement detected at the third interface.

Example 2. The control system of any one of the preceding examples, but particularly Example 1, wherein the handle grip is pivotally attached to the base member.

Example 3. The control system of any one of the preceding examples, but particularly Example 1, wherein the first interface is a handle configured to receive a grip of a user, the first interface being configured to receive a first input to position the medical device relative to a remote center of motion of the medical device.

Example 4. The control system of any one of the preceding examples, but particularly Example 3, wherein the first input comprises a manual input configured to provide movement of the control system and the coupled medical device without engaging electromechanical components of the control system.

Example 5. The control system of any one of the preceding examples, but particularly Example 1, wherein the second interface further comprises an electromechanical controller subsystem configured to receive and translate a plurality of inputs to articulate the end-effector about an end-effector pitch axis, an end-effector yaw axis, and an end-effector roll axis.

Example 6. The control system of any one of the preceding examples, but particularly Example 5, wherein the second interface comprises a joystick finger interface.

Example 7. The control system of any one of the preceding examples, but particularly Example 5, wherein the plurality of inputs comprise motions received at the second interface, the motions being performed by fingers of a user to engage electromechanical components to orient the end-effector.

Example 8. The control system of any one of the preceding examples, but particularly Example 1, wherein the third interface is a compressible finger interface configured to receive and translate a trigger input to control the function of the end-effector.

Example 9. The control system of any one of the preceding examples, but particularly Example 8, wherein the trigger input comprises one or more finger inputs received at the compressible finger interface, the one or more finger inputs being configured to engage additional electromechanical components to perform a predefined function of the end-effector.

Example 10. The control system of any one of the preceding examples, but particularly Example 1, wherein the third interface is a compressible finger interface configured to receive and translate a trigger input to maintain a state of the end-effector.

Example 11. The control system of any one of the preceding examples, but particularly Example 1, wherein the first interface is a mechanical interface for a forearm and hand of a user, the first interface being configured to receive a first input to position the medical device relative to a remote center of motion.

Example 12. The control system of any one of the preceding examples, but particularly Example 1, wherein the second interface is a ringed finger interface, the second interface further comprising an electromechanical controller subsystem configured to receive and translate a plurality of inputs to articulate the end-effector about an end-effector pitch axis, an end-effector yaw axis, and an end-effector roll axis.

Example 13. The control system of any one of the preceding examples, but particularly Example 1, wherein the sensor assembly is configured to generate a corresponding sensor signal based on a detected input at the second interface or a detected input at the third interface, and wherein the control system further comprises: a controller communicatively coupled to the sensor assembly to receive the corresponding sensor signal and to generate a corresponding primary control signal; and a powered actuation unit communicatively coupled to the controller to receive each primary control signal and configured to actuate the end-effector of the medical device received in the device attachment unit based on each primary control signal.

Example 14. The control system of any one of the preceding examples, but particularly Example 1, wherein: the second interface is engageable by a first set of fingers of a user; the third interface is engageable by a second set of fingers of the user; and the second interface and the third interface provide four degrees of freedom of movement of the medical device relative to the base member pivotally connected to the first interface.

Example 15. The control system of any one of the preceding examples, but particularly Example 14, wherein at least one finger in the first set of fingers is also in the second set of fingers.

Example 16. The control system of any one of the preceding examples, but particularly Example 1, wherein the medical device is a surgical device having an elongate shaft extending from a distal tip comprising the end-effector.

Example 17. The control system of any one of the preceding examples, but particularly Example 1, wherein the control system is removably attached to a stabilizing apparatus configured to at least partially support a weight of the control system and define a remote center of motion, the stabilizing apparatus having a remote base configured to be fixed relative to a patient support apparatus and having a pivotable arm attached to the device attachment unit, wherein the device attachment unit is movable relative to the remote base and configured to removably receive the control system.

Example 18. The control system of any one of the preceding examples, but particularly Example 1, wherein the control system is a handle assembly configured for installation in a hybrid, direct-control and robotic-assisted surgical system.

Example 19. The control system of any one of the preceding examples, but particularly Example 1, wherein the sensor assembly comprises at least one potentiometer or encoder to detect an orientation of the second interface and third interface about at least one of a pitch, roll and yaw axes.

Example 20. The control system of any one of the preceding examples, but particularly Example 1, wherein the handle grip is movable relative to the device attachment unit about at least a first degree of freedom and the first interface determines an orientation of the handle grip about the first degree of freedom.

Example 21. The control system of any one of the preceding examples, but particularly Example 1, wherein control of the orientation of the end-effector is control of multiple degrees of freedom of the end-effector about a wrist joint of the end-effector.

Example 22. A method of performing a surgical procedure, the method comprising: actuating, at a first interface of a control system, an input control to position a medical device relative to a remote center of motion; actuating, at a second interface, a first portion of a handle grip configured to control an orientation of an end-effector of the medical device, the second interface being communicatively coupled to a sensor assembly to monitor movement detected at the second interface; and actuating, at a third interface, a second portion of the handle grip configured to control a function of the end-effector, the third interface being communicatively coupled to the sensor assembly to monitor movement detected at the third interface.

Example 23. The method of any one of the preceding examples, but particularly Example 22, wherein actuating the input control comprises maneuvering the first interface using one or both of a forearm and a wrist to cause movement of the medical device about an instrument roll axis, such that the forearm or wrist align with a center of the instrument roll axis.

Example 24. The method of any one of the preceding examples, but particularly Example 22, wherein actuating the first portion at the second interface comprises gripping the first portion of the handle grip with a thumb and a finger.

Example 25. The method of any one of the preceding examples, but particularly Example 22, wherein actuating the second portion at the third interface comprises gripping the second portion of the handle grip with one or more fingers.

Example 26. The method of any one of the preceding examples, but particularly Example 22, wherein actuation of each of the input control, the first portion, and the second portion is mapped to a separate hand portion or body portion.

Example 27. A control system for a medical device, the control system comprising: a first interface configured to control a position of the medical device, the first interface comprising a handle grip coupled to a base member, the base member being coupled to a device attachment unit configured to couple to the medical device; a second interface installed in a first portion of the handle grip and configured to control an orientation of an end-effector of the medical device; and a third interface installed in a second portion of the handle grip and configured to control a function of the end-effector.

Example 28. A control system for a medical device, the control system comprising: a first interface configured to control a position of the medical device, the first interface comprising a handle grip coupled to a base member, the base member being coupled to a device attachment unit configured to couple to the medical device; and a second interface installed in the handle grip and configured to control an orientation of an end-effector of the medical device and a function of the end-effector.

Example 29. A control system for a medical device, the control system comprising: a first interface configured to control a position of the medical device, the first interface comprising a handle grip coupled to a base member; a second interface installed in a first portion of the handle grip and configured to control an orientation of an end-effector of the medical device, the second interface being communicatively coupled to a sensor assembly to monitor movement detected at the second interface; and a third interface installed in a second portion of the handle grip and configured to control a function of the end-effector, the third interface being communicatively coupled to the sensor assembly to monitor movement detected at the third interface.

1. A control system for a medical device, the control system comprising:
a first interface configured to control a position of the medical device, the first interface comprising a handle grip coupled to a base member, the base member being coupled to a device attachment unit configured to couple to the medical device;
a second interface installed in a first portion of the handle grip and configured to control an end-effector orientation of an end-effector of the medical device, the second interface being communicatively coupled to a sensor assembly to monitor movement detected at the second interface; and
a third interface installed in a second portion of the handle grip and configured to control a function of the end-effector, the third interface being communicatively coupled to the sensor assembly to monitor movement detected at the third interface.

2. The control system of claim 1, wherein the handle grip is pivotally attached to the base member.

3. The control system of claim 1, wherein the first interface is a handle configured to receive a grip of a user, the first interface being configured to receive a first input to position the medical device relative to a remote center of motion of the medical device, and wherein the first input comprises a manual input configured to provide movement of the control system and the coupled medical device without engaging electromechanical components of the control system.

4. (canceled)

5. The control system of claim 1, wherein the second interface further comprises an electromechanical controller subsystem configured to receive and translate a plurality of inputs to articulate the end-effector about an end-effector pitch axis, an end-effector yaw axis, and an end-effector roll axis.

6. The control system of claim 5, wherein the second interface comprises a joystick finger interface.

7. The control system of claim 5, wherein the plurality of inputs comprise motions received at the second interface, the motions being performed by fingers of a user to engage electromechanical components to orient the end-effector.

8. The control system of claim 1, wherein the third interface is a compressible finger interface configured to receive and translate a trigger input to control the function of the end-effector, and wherein the trigger input comprises one or more finger inputs received at the compressible finger interface, the one or more finger inputs being configured to engage additional electromechanical components to perform a predefined function of the end-effector.

9. (canceled)

10. The control system of claim 1, wherein the third interface is a compressible finger interface configured to receive and translate a trigger input to maintain a state of the end-effector.

11. The control system of claim 1, wherein the first interface is a mechanical interface for a forearm and hand of a user, the first interface being configured to receive a first input to position the medical device relative to a remote center of motion.

12. The control system of claim 1, wherein the second interface is a roll barrel interface, the second interface further comprising an electromechanical controller subsystem configured to receive and translate an input to articulate the end-effector about an end-effector roll axis.

13. The control system of claim 1, wherein the sensor assembly is configured to generate a corresponding sensor signal based on a detected input at the second interface or a detected input at the third interface, and wherein the control system further comprises:
a controller communicatively coupled to the sensor assembly to receive the corresponding sensor signal and to generate a corresponding primary control signal; and
a powered actuation unit communicatively coupled to the controller to receive each primary control signal and configured to actuate the end-effector of the medical device received in the device attachment unit based on each primary control signal.

14. The control system of claim 1, wherein:
the second interface is engageable by a first set of fingers of a user;
the third interface is engageable by a second set of fingers of the user; and
the second interface and the third interface provide four degrees of freedom of movement of the medical device relative to the base member pivotally connected to the first interface.

15. The control system of claim 14, wherein at least one finger in the first set of fingers is also in the second set of fingers.

16. The control system of claim 1, wherein the medical device is a surgical device having an elongate shaft extending from a distal tip comprising the end-effector.

17. The control system of claim 1, wherein the control system is removably attached to a stabilizing apparatus configured to at least partially support a weight of the control system and define a remote center of motion, the stabilizing apparatus having a remote base configured to be fixed relative to a patient support apparatus and having a pivotable arm attached to the device attachment unit, wherein the device attachment unit is movable relative to the remote base and configured to removably receive the control system.

18. The control system of claim 1, wherein the control system is a handle assembly configured for installation in a hybrid, direct-control and robotic-assisted surgical system.

19. The control system of claim 1, wherein the sensor assembly comprises at least one potentiometer or encoder to detect an orientation of the second interface and third interface about at least one of a pitch, roll and yaw axes.

20. The control system of claim 1, wherein the handle grip is movable relative to the device attachment unit about at least a first degree of freedom and the first interface determines an orientation of the handle grip about the first degree of freedom.

21. The control system of claim 1, wherein control of the orientation of the end-effector is control of multiple degrees of freedom of the end-effector about a wrist joint of the end-effector.

22. A method of performing a surgical procedure, the method comprising:
actuating, at a first interface of a control system, an input control to position a medical device relative to a remote center of motion;
actuating, at a second interface, a first portion of a handle grip configured to control an orientation of an end-effector of the medical device, the second interface being communicatively coupled to a sensor assembly to monitor movement detected at the second interface; and
actuating, at a third interface, a second portion of the handle grip configured to control a function of the end-effector, the third interface being communicatively coupled to the sensor assembly to monitor movement detected at the third interface.

23. The method of claim 22, wherein actuating the input control comprises maneuvering the first interface using one or both of a forearm and a wrist to cause movement of the medical device about an instrument roll axis, such that the forearm or wrist align with a center of the instrument roll axis.

24. The method of claim 22, wherein actuating the first portion at the second interface comprises gripping the first portion of the handle grip with a thumb and a finger.

25. The method of claim 22, wherein actuating the second portion at the third interface comprises gripping the second portion of the handle grip with one or more fingers.

26. The method of claim 22, wherein actuation of each of the input control, the first portion, and the second portion is mapped to a separate hand portion or body portion.

27. (canceled)

28. (canceled)

29. (canceled)
